SEQUENCE LISTING (8 sequences)

SEQ ID NO: 1 - protein, 647 aa, Homo sapiens
MPLVDFFCETCSKPWLVGWWDQFKRMLNRELTHLSEMSRSGNQVSEYISTTFLDKQNEVEIPSPTMKEREKQQAPRPRPSQPPPPPVPHLQPMSQITGLKKLMHSNSLNNSNIPRFGVKTDQEELLAQELENLNKWGLNIFCVSDYAGGRSLTCIMYMIFQERDLLKKFRIPVDTMVTYMLTLEDHYHADVAYHNSLHAADVLQSTHVLLATPALDAVFTDLEILAALFAAAIHDVDHPGVSNQFLINTNSELALMYNDESVLENHHLAVGFKLLQEDNCDIFQNLSKRQRQSLRKMVIDMVLATDMSKHMTLLADLKTMVETKKVTSSGVLLLDNYSDRIQVLRNMVHCADLSNPTKPLELYRQWTDRIMAEFFQQGDRERERGMEISPMCDKHTASVEKSQVGFIDYIVHPLWETWADLVHPDAQEILDTLEDNRDWYYSAIRQSPSPPPEEESRGPGHPPLPDKFQFELTLEEEEEEEISMAQIPCTAQEALTAQGLSGVEEALDATIAWEASPAQESLEVMAQEASLEAELEAVYLTQQAQSTGSAPVAPDEFSSREEFVVAVSHSSPSALALQSPLLPAWRTLSVSEHAPGLPGLPSTAAEVEAQREHQAAKRACSACAGTFGEDTSALPAPGGGGSGGDPT

SEQ ID NO: 2 - protein, 25 aa, Homo sapiens
SLLTNVPVPSNKRSPLGGPTPVCKA

SEQ ID NO: 3 - protein, 25 aa, Homo sapiens
VPVPSNKRSPLGGPTPVCKATLSEE

SEQ ID NO: 4 - protein, 25 aa, Homo sapiens
TLEDNRDWYYSAIRQSPSPPPEEES

SEQ ID NO: 5 - protein, 25 aa, Homo sapiens
RDWYYSAIRQSPSPPPEEESRGPGH

SEQ ID NO: 6 - protein, 25 aa, Homo sapiens
KRACSACAGTFGEDTSALPAPGGGG

SEQ ID NO: 7 - protein, 25 aa, Homo sapiens
ACAGTFGEDTSALPAPGGGGSGGDP

SEQ ID NO: 8 - protein, 36 aa, Homo sapiens
YGRKKRRQRRRSPLDSQASPGLVLHAGATTSQRRES

(19) United States
(12) Patent Application Publication
(10) Pub. No.: US 2010/0216703 A1
(43) Pub. Date: Aug. 26, 2010

(54) Inhibitors of PDE4 and Methods of Use

(75) Inventors: Katerina Akassoglou, San Francisco, CA (US); Miles D. Houslay, Renfrewshire (GB); Moses V. Chao, New York, NY (US)

Correspondence Address: SPENCER FANE BRITT & BROWNE LLP, 1 NORTH BRENTWOOD BLVD., SUITE 1000, ST. LOUIS, MO, 63105-3925, US

(73) Assignees: THE REGENTS OF THE UNIVERSITY OF CALIFORNIA, Oakland, CA (US); NEW YORK UNIVERSITY, New york, NY (US); UNIVERSITY OF GLASGOW, Glasgow, Scotland (GB)

(21) Appl. No.: 12/377,478

(22) PCT Filed: Aug. 14, 2007
(86) PCT No.: PCT/US2007/075934

§ 371 Date: May 12, 2010

Related U.S. Application Data
(60) Provisional application No. 60/837,542, filed on Aug. 14, 2006.

(51) Int. Cl. A61K 38/17; C07K 14/435; C12Q 1/02; A61P 11/00

(52) U.S. Cl. 514/12; 530/324; 435/29

(57) ABSTRACT

The inventors have succeeded in discovering that the p75 neurotrophin receptor (p75NTR) is directly involved in the degradation of cAMP via interaction of its intracellular domain with phosphodiesterase 4A4/5 (PDE4A4/5). Provided herein are methods and compositions for the treatment of conditions of PDE4A4/5 and p75NTR expression (such as pulmonary disease and nerve regeneration) by blocking the interaction of PDE4A4/5 and p75NTR, as well as methods for the screening of agents useful in such applications.

### CROSS-REFERENCE TO RELATED APPLICATIONS

This application claims priority from U.S. Provisional Application Ser. No. 60/837,542 filed on Aug. 14, 2006, which is incorporated herein by reference in its entirety.

### STATEMENT REGARDING FEDERALLY SPONSORED RESEARCH OR DEVELOPMENT

This invention was made in part with Government support under National Institutes of Health Grant NS051470. The Government has certain rights in the invention.

### INCORPORATION-BY-REFERENCE OF MATERIAL SUBMITTED ON A COMPACT DISC

The Sequence Listing, which is a part of the present disclosure, includes a computer file “10100_{—}0080_ST25.TXT” generated by U.S. Patent & Trademark Office Patentln Version 3.4 software comprising nucleotide and/or amino acid sequences of the present invention. The subject matter of the Sequence Listing is incorporated herein by reference in its entirety.

### FIELD

The present invention generally relates to specific inhibitors of PDE4A4 and uses thereof.

### INTRODUCTION

Current inhibitors of phosphodiesterases, and especially PDE4, non-selectively inhibit all the PDE4 isoforms resulting in various side effects, such as emesis. Such inhibitors include rolipram (Schering) and atizoram (Pfizer) (see Houslay et al., describing known inhibitors). While these inhibitors are currently in clinical trials for asthma and COPD, several of these trials were discontinued due to the side effects of non-selective PDE4 inhibition.

Tissue scarring, characterized by cell activation, excessive deposition of extracellular matrix and extravascular fibrin deposition, is considered a limiting factor for tissue repair. Fibrin is a provisional matrix deposited after vascular injury and is the major substrate of plasmin (Bugge et al., 1996). Local generation of plasmin is regulated by two Plasminogen Activators (PAs): the serine proteases tissue type PA (tPA) and urokinase type PA (uPA) (Lijnen, 2001). PAs and their inhibitors are key modulators of scar resolution by spatially and temporally regulating the conversion of plasminogen to plasmin resulting in fibrin degradation and extracellular matrix remodeling. Studies of fibrin deposition in human diseases, in combination with experiments from gene-targeted mice deficient in plasminogen and PAs (Degen et al., 2001), have provided information about a wide range of physiological and pathological conditions that are exacerbated by defective fibrin degradation, such as wound healing, metastasis, atherosclerosis, lung ischemia, rheumatoid arthritis, muscle and nerve regeneration and multiple sclerosis.

Extracellular matrix remodeling regulates a variety of nervous system functions, such as neuronal development, regeneration and synaptic plasticity (Dityatev and Schachner, 2003). Fibrin is a component of the extracellular matrix during injury and in diseases associated with vascular damage and leakage of the blood-brain barrier (BBB), such as multiple sclerosis, stroke and sciatic nerve injury (for review see (Adams et al., 2004)). In the nervous system mice deficient in plasminogen or tPA show exacerbated axonal damage (Akassoglou et al., 2000) and impaired functional recovery (Siconolfi and Seeds, 2001) after sciatic nerve injury. In accordance, mice deficient for fibrinogen show increased regenerative capacity (Akassoglou et al., 2002). In the central nervous system 4 (CNS) genetic or pharmacologic depletion of fibrin delays the onset of inflammatory demyelination in an animal model of multiple sclerosis (MS) (Akassoglou et al., 2004). MS demyelinated plaques show impaired fibrinolysis suggesting that regulation of the tPA/plasmin system is affected in MS lesions (Gveric et al., 2003). Indeed, depletion of tPA exacerbates the disease (Lu et al., 2002). Overall, these studies suggest that regulation of proteolytic activity determines fibrin clearance and regulates the extent of damage and the recovery potential of the nervous system from injury. However, the molecular mechanisms that the nervous system utilizes to regulate proteolytic activity remain unclear.

It has been demonstrated that fibrin regulates expression of p75 neurotrophin receptor (p75NTR) after nerve injury (Akassoglou et al., 2002). Upregulation of p75NTR is frequently observed in multiple sclerosis (Chang et al., 2000; Dowling et al., 1999), stroke (Park et al., 2000), spinal cord (Beattie et al., 2002) and sciatic nerve injury (Taniuchi et al., 1986); all of which are associated with BBB disruption and fibrin deposition. In addition to the nervous system, p75NTR is expressed in non-neuronal tissues (Lomen-Hoerth and Shooter, 1995) and is upregulated in a variety of diseases associated with defects in fibrin degradation, such as atherosclerosis (Wang et al., 2000), pancreatitis (Zhu et al., 2003), melanoma formation (Herrmann et al., 1993), lung inflammation (Renz et al., 2004), cancer (Krygier and Djakiew, 2001) and liver disease (Cassiman et al., 2001). p75NTR has been primarily characterized as a modulator of cell death in non-neuronal tissues (Kraemer, 2002; Wang et al., 2000). The expression of p75NTR by cell types such as smooth muscle cells and hepatic stellate cells, that actively participate in tissue repair by migration, secretion of ECM and extracellular 5 proteases, raises the possibility for a functional role of p75NTR in disease pathogenesis that extends beyond apoptosis and proliferation.

Identification of specific targeting of phosphodiesterase isoforms has long been sought. But available chemical inhibitors, such as rolipram, inhibit all twenty isoforms of the PDE4 subfamily.

### SUMMARY

The inventors have succeeded in discovering that the p75 neurotrophin receptor (p75NTR) is directly involved in the degradation of cAMP via interaction of its intracellular domain with phosphodiesterase 4A4/5 (PDE4A4/5).

Among the various aspects of the present invention is the provision of a method of treating a condition resulting from PDE4A4/5-mediated cAMP degradation. Such method includes the step of administering to a subject in need thereof a therapeutically effective amount of an agent that disrupts the interaction between PDE4A4/5 and p75 neurotropin receptor (p75NTR). The condition treated is, for example, a pulmonary disease or nerve injury, more specifically COPD or spinal cord injury.

Another aspect of the invention includes an isolated polypeptide, derived from PDE4A4/5, with the ability to specifically block the molecular interaction between p75NTR and PDE4A4/5. Such polypeptides include, for example, those comprising sequences of SEQ ID NO: 2, SEQ ID NO: 3, SEQ ID NO: 4, SEQ ID NO: 5, SEQ ID NO: 6, or SEQ ID NO: 7. Such polypeptides also include, for example, variants at least 80% identical to sequences SEQ ID NO: 2, SEQ ID NO: 3, SEQ ID NO: 4, SEQ ID NO: 5, SEQ ID NO: 6, or SEQ ID NO: 7. The polypeptide can be an isolated polypeptide according to claim 6, wherein the polypeptide specifically binds amino acid C862.

Another aspect of the invention includes an isolated polypeptide comprising a sequence at least 80% identical to a subunit of PDE4A4/5 that interacts with p75NTR and having an ability to specifically block the molecular interaction between p75NTR and PDE4A4/5. Such PDE4A4/5 subunits include, for example, the LR1, catalytic, or C-terminus subunits of PDE4A4/5.

Another aspect of the invention provides a method of screening an agent for treating a disease resulting from PDE4A4/5-mediated cAMP degradation. Such method includes the steps of providing a cell that stably expresses PDE4A4/5 and p75NTR; administering a candidate agent to the cell; measuring a level of PDE4A4/5-p75NTR complex in the cell; and determining whether the candidate agent decreases the level of PDE4A4/5-p75NTR complex in the cell. In another aspect, the method can include the steps of providing PDE4A4/5 and p75NTR; contacting a candidate agent, PDE4A4/5, and p75NTR; measuring a level of PDE4A4/5-p75NTR complex; and determining whether the candidate agent decreases the level of PDE4A4/5-p75NTR complex.

These and other features, aspects and advantages of the present teachings will become better understood with reference to the following description, examples and appended claims.

### DRAWINGS

Those of skill in the art will understand that the drawings, described below, are for illustrative purposes only. The drawings are not intended to limit the scope of the present teachings in any way.

FIG. 1. Series of panels illustrating immunohistochemistry and Western blot results showing that fibrin deposition is reduced in the sciatic nerve of p75^{NTR}−/− mice.

FIG. 2. Series of panels illustrating in situ fibrin zymography and double immunofluorescence results showing that p75^{NTR} regulates expression of tPA in the sciatic nerve after crush injury.

FIG. 3: Panel series illustrating results of analysis of primary Schwann cell cultures showing that p75^{NTR} mediates regulation of tPA and fibrinolysis in Schwann cells.

FIG. 4: Series of panels illustrating results of analysis of NIH3T3 cells in culture, bar graphs and immunoblot analysis showing that expression of p75^{NTR} regulates tPA, PAI-1 and fibrinolysis in fibroblasts.

FIG. 5: Series of panels illustrating cell culture and PKA activity assay results showing that p75^{NTR} Regulates tPA and PAI-1 via a PDE4/cAMP/PKA Pathway.

FIG. 6: Series of panels illustrating various assay results and schematic diagrams showing that p75^{NTR} directly interacts with PDE4A5.

FIG. 7: Series of panels illustrating levels of fibrin deposition, and Western blot analysis, in lungs of wt and lipopolysaccharide (LPS)-induced fibrotic mice showing that p75^{NTR} regulates fibrin clearance in the lung.

FIG. 8: Schematic diagram illustrating proposed model for the role of p75^{NTR} in the cAMP-mediated plasminogen activation.

FIG. 9: Series of panels illustrating results of fibrin immunostainings, zymographies and quantitative analysis of results showing that loss of tPA rescues the effects of p75^{NTR} deficiency in plasminogen activation and fibrin deposition in the sciatic nerve.

FIG. 10. Series of panels illustrating results of real time PCR analysis and in situ zymographies on cerebella from wild-type and p75^{NTR}−/− mice showing that loss of p75^{NTR} leads to an increase in tPA mRNA levels and proteolytic activity in the central nervous system.

FIG. 11: Panels A-B illustrating results of endogenous coimmunoprecipitation between p75^{NTR} and PDE4A5 in freshly isolated Cerebellar Granule Neurons (CGN) and in injured sciatic nerve, showing that endogeneous levels of PDE4A5 and p75^{NTR} are able to form a complex in wt CGN and in injured sciatic nerve. Western blot results show similar levels of PDE4A5 expression on NIH3T3 and NIH3T3 p75^{NTR} cells (C)

FIG. 12: Series of panels illustrating schematic illustration of generation of PKA fluorescent indicator (A) and results (B-E) of analysis of p75^{NTR}-mediated inhibition of cAMP using FRET-based PKA biosensors.

FIG. 13: Computational docking of the catalytic subunit of PDE4A4 with the intracellular domain of p75^{NTR}.

FIG. 14: Series of panels showing multiple cAMP assay and immunohistochemical results demonstrating that p75^{NTR} down regulates cAMP by targeting its degradation to the plasma membrane.

FIG. 15: Series of panels showing co-immunoprecipitation results and related schematic diagram demonstrating that p75^{NTR} co-immunoprecipitates with PDE4A5 and the p75^{NTR} juxtamembrane sequence (Arg275-Leu342) associates with PDE4A5.

FIG. 16: Series of panels illustrating steps involved in mapping the p75^{NTR}. PDE4A4 sequences that interact with p75^{NTR}.

FIG. 17: Series of panels showing that block of the PDE4A-p75^{NTR} interaction with synthetic peptides designed to competitively inhibit the interaction between PDE4A4 and p75^{NTR} (peptides 136 and 172), overcomes myelin inhibition of neurite outgrowth in CGN.

FIG. 18: Series of panels showing analysis of PDE4A4 domains and interacting sequences of PDE4A4.

FIG. 19. Bar graph showing quantitative analysis of intracellular cAMP showing that p75^{NTR} decreases intracellular cAMP in a neurotrophin dependent manner.

FIG. 20. Series of panels showing fibrin deposition assay and quantitative PCR results showing that rolipram decreases fibrin deposition both in LPS-induced lung fibrosis and sciatic nerve crush injuries.

FIG. 21. Series of panels showing cAMP assay results showing that p75^{NTR} decreases intracellular cAMP via PDE4.

### DETAILED DESCRIPTION

### PDE4 Inhibitors and Methods of Use

The present invention provides methods and compositions for the treatment of conditions of PDE4A4/5 and p75 neurotropin receptor (p75NTR) expression (such as pulmonary disease and nerve regeneration) by blocking the interaction of PDE4A4/5 and p75NTR, as well as methods for the screening of agents useful in such applications.

The technology described herein is based in part on the observation of a novel molecular interaction between p75NTR and phosphodiesterases; and p75NTR is directly involved in the degradation of cAMP via interaction of its intracellular domain with PDE4A4/5. As such, the p75NTR-PDE4A4/5 complex presents a therapeutic target for conditions associated with PDE4A4/5-mediated cAMP degradation. Such conditions include pulmonary disease (e.g., pulmonary fibrosis) and nerve injury (e.g., axonal regeneration). The studies reported here identify p75NTR as a regulator of proteolytic activity and fibrin degradation during peripheral nerve regeneration and pulmonary fibrosis via directly binding to phosphodiesterases and decreasing intracellular cAMP. Data disclosed herein show for the first time that plasminogen activation is down-regulated by a neurotrophin receptor via a cAMP/PKA mechanism, p75NTR induces degradation of cAMP, and phosphodiesterases can be recruited to the membrane via direct binding to a transmembrane receptor.

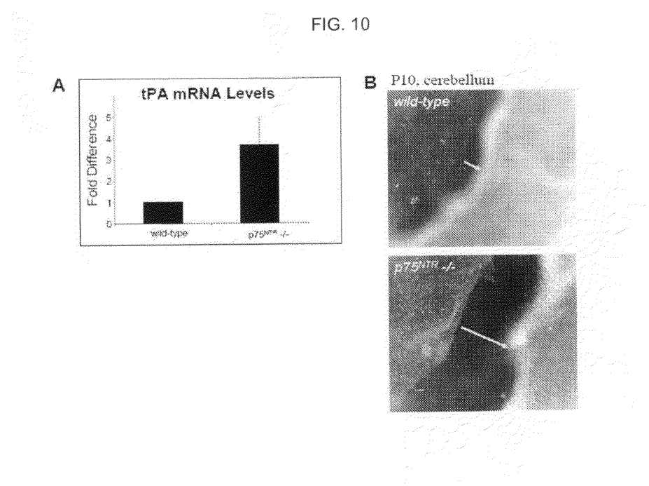

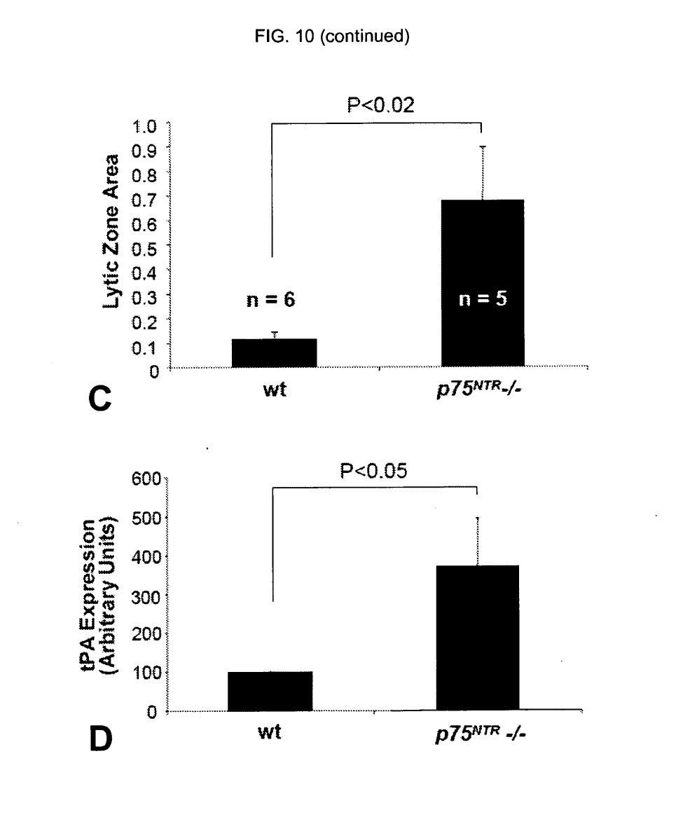

Without being bound by a particular theory, it is thought that p75NTR has the following role in the regulation of plasminogen activation (see e.g., FIG. 10). Injury induces upregulation of p75NTR in a variety of cell types within and outside of the nervous system. p75NTR directly interacts with (i.e., recruits) PDE4A5 and induces degradation of cAMP resulting in decreased PKA activity. Downregulation of cAMP induces upregulation of PAI-1 and downregulation of tPA resulting in decreased extracellular proteolysis. Decreased proteolytic activity inhibits extracellular matrix remodeling and fibrinolysis in the sciatic nerve and the lung.

Three binding motifs of PDE4A5, within the LR1, catalytic, and C-terminal subunits, mediate recruitment of p75NTR to the membrane (see e.g., Example 6, Example 11). The LR1 domain is unique for the PDE4A subfamily. In addition, the C-terminal domain is unique for each PDE4 subfamily. The extreme C-terminus of PDE4A5 is the major interacting domain with p75NTR, demonstrating its role as a regulator of isoform-specific phosphodieterase recruitment to subcellular locations. Thus, PDE4A5 is a molecular mediator of p75NTR/cAMP signaling that regulates plasminogen activation and fibrinolysis.

### Treatment

One aspect of the invention provides methods of treatment for conditions related to, or exacerbated by, PDE4A4/5 and p75NTR expression and/or PDE4A4/5-mediated cAMP degradation. P75NTR is directly involved in the degradation of cAMP via interaction of its intracellular domain with PDE4A4/5. As described herein, mediation of p75NTR activity can regulate disease progression via accumulation of plasmin-cleaved substrates in both neuronal and non-neuronal tissues. Conditions resultant from cAMP degradation can, therefore, be treated in a subject in need thereof by administering an agent that down regulates p75NTR and/or interferes with p75NTR interaction with PDE4A5.

One aspect of the invention provides a method of treating a condition related to expression and/or activity of PDE4A4/5 and p75NTR expression. Such conditions may result from cAMP degradation. The treatment method involves administering to a subject in need thereof an agent that disrupts the interaction between PDE4A4/5 and p75 neurotropin receptor (p75NTR). Disruption of the molecular interaction between p75NTR and PDE4A4/5 can increase tPA activity, decrease fibrin levels, increase finbrin degradation, increase extracellular proteolysis, decrease degradation of cAMP by phosphodiesterase, and/or increase PKA activity. For example, Tat-fused PDE4A4 peptide sequences that interact with p75NTR rescue the myelin-induced, p75NTR-mediated inhibition of neurite outgrowth.

Disease states or conditions indicative of a need for therapy in the context of the present invention, and/or amenable to treatment methodologies described herein, include any condition caused by, or exacerbated by, PDE4A4/5 and p75NTR expression and/or PDE4A4/5-mediated cAMP degradation, such as pulmonary disease (e.g., asthma and COPD), nerve regeneration (e.g., spinal cord injury), tissue scarring, wound healing, metastasis, atherosclerosis, lung ischemia, rheumatoid arthritis, muscle and nerve regeneration, stroke, multiple sclerosis, pancreatitis, melanoma formation, lung inflammation, cancer, liver disease, inflammatory bowel disease, and depression and/or mood disorders. Such conditions can be those exacerbated by defective fibrin degradation.

A determination of the need for treatment will typically be assessed by a history and physical exam consistent with the condition. Such diagnosis is within the skill of the art. Subjects with an identified need of therapy include those with a diagnosed condition described herein or indication of a condition amenable to therapeutic treatment described herein and subjects who have been treated, are being treated, or will be treated for such conditions. The subject is preferably an animal, including, but not limited to, mammals, reptiles, and avians, more preferably horses, cows, dogs, cats, sheep, pigs, and chickens, and most preferably human.

### Compositions

Another aspect of the invention provides agents that block the molecular interaction between p75NTR and phosphodiesterases, especially the PDE4A4/5 isoforms. Such agent is relevant to a variety of applications, including therapeutic applications directed towards conditions associated with expression of p75NTR and PDE4A4/5, such as nerve regeneration and pulmonary fibrosis.

As described herein, p75NTR regulates proteolytic activity and fibrin degradation during peripheral nerve regeneration and pulmonary fibrosis via directly binding to phosphodiesterases and decreasing intracellular cAMP. Provided herein are agents that can effect proteolytic activity, fibrin degradation, and cAMP levels through their ability to specifically block the interaction between p75NTR and PDEA4/5. Preferably, such agent is specific for the PDEA4/5 isoforms and does not interfere with the activity of other phosphodiesterase isoforms.

The various classes of agents for use herein as agents that specifically block the molecular interaction between p75NTR and PDE4A4/5, generally include, but are not limited to, peptides, RNA interference molecules, antibodies, small inorganic molecules, antisense oligonucleotides, and aptamers.

### Peptides

Included within the scope of the invention are peptide molecules that specifically interact with p75NTR and/or PDE4A4/5 (SEQ ID NO: 1; GenBank Accession No. NP_{—}006193) and can be used to specifically block the molecular interaction between p75NTR and PDE4A4/5. It is shown herein that Tat-fused PDE4A4 peptide sequences that interact with p75NTR rescue the myelin-induced, p75NTR-mediated inhibition of neurite outgrowth (see e.g., Example 12).

Such polypeptide can be derived from PDE4A4/5 and/or p75NTR, or particular subunits of PDE4A4/5 that interact with p75NTR, and vice versa. For example, such polypeptides can be derived from the LR1, catalytic, or C-terminus subunits of PDE4A4 that bind to the intracellular domain of p75NTR. Peptide sequences derived from the LR1, catalytic, and C-terminus subunits of PDE4A4 that bind to the intracellular domain of p75NTR can be used to block the molecular interaction between p75NTR and PDE4A4/5 (see e.g., Example 13). The following discussion focuses upon peptides derived from the PDE4A4/5 protein, but one skilled in the art will understand that such discussion applies equally to peptides derived from p75NTR protein.

Polypeptides of the invention include those variants of native PDE4A4/5 proteins such as fragments, analogs and derivatives of native PDE4A4/5 proteins that have the ability to specifically block the molecular interaction between p75NTR and PDE4A4/5. PDE4A4/5 protein fragment variants have a peptide sequence that differs from the corresponding native PDE4A4/5 protein fragment in one or more amino acids. The peptide sequence of such variants can feature a deletion, addition, or substitution of one or more amino acids of a native PDE4A4/5 polypeptide, or fragment thereof. Amino acid insertions are preferably of about 1, 2, 3, and 4 to 5 contiguous amino acids, and deletions are preferably of about 1, 2, 3, 4, 5, 6, 7, 8, and 9 to 10 contiguous amino acids.

PDE4A4/5 protein fragments corresponding to one or more particular motifs and/or domains or to arbitrary sizes, for example, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 75, 100, 125, 150, 200, 250, 300, 400, 500, and 600 amino acids in length are intended to be within the scope of the present invention. Isolated peptidyl portions of PDE4A4/5 proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a PDE4A4/5 protein of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a p75NTR-PDE4A4/5 complex.

Polypeptides of the invention also include those polypeptides having the ability to specifically block the molecular interaction between p75NTR and PDE4A4/5 and at least 80% sequence identity to PDE4A4 and/or PDE4A4/5, or a portion thereof. For example, inhibitory peptides can have 81%, 82%, 83%, 84%, 85%, 86%, 87%, 88%, 89%, 90%, 91%, 92%, 93%, 94%, 95%, 96%, 97%, 98%, or 99% sequence identity to PDE4A4 and/or PDE4A4/5. Such molecules can include, for example, SEQ ID NO: 2, SEQ ID NO: 3, SEQ ID NO: 4, SEQ ID NO: 5, SEQ ID NO: 6, and SEQ ID NO: 7. In addition, such molecules include polypeptides having longer or shorter amino acid sequences and having the ability to specifically block the molecular interaction between p75NTR and PDE4A4/5.

As used herein, “sequence identity” means the percentage of identical subunits at corresponding positions in two sequences when the two sequences are aligned to maximize subunit matching, i.e., taking into account gaps and insertions. Sequence identity is present when a subunit position in both of the two sequences is occupied by the same nucleotide or amino acid, e.g., if a given position is occupied by an adenine in each of two DNA molecules, then the molecules are identical at that position. For example, if 7 positions in a sequence 10 nucleotides in length are identical to the corresponding positions in a second 10-nucleotide sequence, then the two sequences have 70% sequence identity. Sequence identity is typically measured using sequence analysis software (e.g., Sequence Analysis Software Package of the Genetics Computer Group, University of Wisconsin Biotechnology Center, 1710 University Avenue, Madison, Wis. 53705).

Proteins that specifically block the molecular interaction between p75NTR and PDE4A4/5 variants can be generated through various techniques known in the art. For example, PDE4A4/5 protein variants can be made by mutagenesis, such as by introducing discrete point mutation(s), or by truncation. Alternatively, antagonistic forms of the protein can be generated which are able to inhibit the function of the naturally occurring form of the protein, such as by competitively binding to another molecule that interacts with PDE4A4/5 protein (e.g., p75NTR). In addition, agonistic forms of the protein may be generated that constitutively express one or more PDE4A4/5 functional activities. Other variants of PDE4A4/5 proteins that can be generated include those that are resistant to proteolytic cleavage, as for example, due to mutations which alter protease target sequences. Whether a change in the amino acid sequence of a peptide results in a PDE4A4/5 protein variant having the ability to specifically block the molecular interaction between p75NTR and PDE4A4/5 can be readily determined by testing.

As another example, proteins that specifically block the molecular interaction between p75NTR and PDE4A4/5 can be generated from a degenerate oligonucleotide sequence derived from PDE4A4/5. Chemical synthesis of a degenerate gene sequence can be carried out in an automatic DNA synthesizer, and the synthetic genes then ligated into an appropriate expression vector. One purpose for a degenerate set of genes is to provide, in one mixture, all of the sequences encoding the desired set of potential protein sequences that may specifically block the molecular interaction between p75NTR and PDE4A4/5. The synthesis of degenerate oligonucleotides is well known in the art (see, e.g., Narang, S A (1983) Tetrahedron 39:3; Itakura et al. (1981) Recombinant DNA, Proc 3rd Cleveland Sympos. Macromolecules, ed. AG Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, e.g., Scott et al. (1990) Science 249:386-390; Roberts et al. (1992) Proc. Natl. Acad. Sci. USA 89:2429-2433; Devlin et al. (1990) Science 249: 404-406; Cwirla et al. (1990) Proc. Natl. Acad. Sci. USA 87: 6378-6382; as well as U.S. Pat. Nos. 5,223,409; 5,198,346; and 5,096,815).

Similarly, a library of coding sequence fragments can be provided for a PDE4A4/5 gene clone in order to generate a variegated population PDE4A4/5 protein fragments for screening and subsequent selection of fragments having the ability to specifically block the molecular interaction between p75NTR and PDE4A4/5. A variety of techniques are known in the art for generating such libraries, including chemical synthesis. In one embodiment, a library of coding sequence fragments can be generated by (i) treating a double-stranded PCR fragment of a PDE4A4/5 gene coding sequence with a nuclease under conditions wherein nicking occurs only about once per molecule; (ii) denaturing the double-stranded DNA; (iii) renaturing the DNA to form double-stranded DNA which can include sense/antisense pairs from different nicked products; (iv) removing single-stranded portions from reformed duplexes by treatment with S1 nuclease; and (v) ligating the resulting fragment library into an expression vector. By this exemplary method, an expression library can be derived which codes for LR1, catalytic, C-terminus, and other terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of PDE4A4/5 gene variants. The most widely used techniques for screening large gene libraries typically involve cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

Combinatorial mutagenesis has a potential to generate very large libraries of mutant proteins. To screen a large number of protein mutants, techniques that allow one to avoid the very high proportion of non-functional proteins in a random library and simply enhance the frequency of functional proteins (thus decreasing the complexity required to achieve a useful sampling of sequence space) can be used. For example, recursive ensemble mutagenesis (REM), an algorithm that enhances the frequency of functional mutants in a library when an appropriate selection or screening method is employed, might be used. Arkin and Yourvan (1992) Proc. Natl. Acad. Sci. USA 89:7811-7815; Yourvan et al. (1992) Parallel Problem Solving from Nature, Maenner and Manderick, eds., Elsevier Publishing Co., Amsterdam, pp. 401-410; Delgrave et al. (1993) Protein Engineering 6(3): 327-331.

The invention also provides for reduction of PDE4A4/5 proteins to generate mimetics, e.g. peptide or non-peptide agents, that are able to disrupt binding of PDE4A4/5 protein to other proteins or molecules, such as p75NTR, with which the native PDE4A4/5 protein interacts. Thus, the techniques described herein can also be used to map which determinants of PDE4A4/5 protein participate in the intermolecular interactions involved in, e.g., binding of PDE4A4/5 protein to other proteins which may function upstream (e.g., activators or repressors of PDE4A4/5 functional activity) of the PDE4A4/5 protein or to proteins or nucleic acids which may function downstream of the PDE4A4/5 protein, and whether such molecules are positively or negatively regulated by the PDE4A4/5 protein. To illustrate, the critical residues of an PDE4A4/5 protein, which are involved in molecular recognition of p75NTR, or other components upstream or downstream of the PDE4A4/5 protein can be determined and used to generate PDE4A4/5 protein-derived peptidomimetics which competitively inhibit binding of the PDE4A4/5 protein to that moiety (see e.g., Example 11). By employing scanning mutagenesis to map the amino acid residues of a PDE4A4/5 protein that are involved in binding other extracellular proteins, peptidomimetic compounds can be generated which mimic those residues of a native PDE4A4/5 protein. Such mimetics may then be used to interfere with the normal function of a PDE4A4/5 protein.

For example, non-hydrolyzable peptide analogs of such residues can be generated using benzodiazepine (see, e.g., Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopepitides (Ewenson et al. (1986) J. Med. Chem. 29:295; and Ewenson et al. in Peptides: Structure and Function (Proceedings of the 9th American Peptide Symposium) Pierce Chemical Co. Rockland, Ill., 1985), beta-turn dipeptide cores (Nagai et al. (1985) Tetrahedron Lett 26:647; and Sato et al. (1986) J. Chem. Soc. Perkin. Trans. 1: 1231), and beta-aminoalcohols (Gordon et al. (1985) Biochem. Biophys. Res. Commun. 126:419; and Dann et al. (1986) Biochem. Biophys. Res. Commun. 134:71). PDE4A4/5 proteins may also be chemically modified to create PDE4A4/5 protein derivatives by forming covalent or aggregate conjugates with other chemical moieties, such as glycosyl groups, lipids, phosphate, acetyl groups and the like. Covalent derivatives of PDE4A4/5 protein can be prepared by linking the chemical moieties to functional groups on amino acid side chains of the protein or at the N-terminus or at the C-terminus of the polypeptide.

The present invention further pertains to methods of producing the subject proteins with the ability to specifically block the molecular interaction between p75NTR and PDE4A4/5. For example, a host cell transfected with a nucleic acid vector directing expression of a nucleotide sequence encoding the subject polypeptides can be cultured under appropriate conditions to allow expression of the peptide to occur. The cells may be harvested, lysed, and the protein isolated. A recombinant PDE4A4/5-derived protein can be isolated from host cells using techniques known in the art for purifying proteins including ion-exchange chromatography, gel filtration chromatography, ultrafiltration, electrophoresis, and immunoaffinity purification with antibodies specific for such protein.

For example, after a PDE4A4/5-derived protein has been expressed in a cell, it can be isolated using any immuno-affinity chromatography. More specifically, a specific antibody can be immobilized on a column chromatography matrix, and the matrix can be used for immuno-affinity chromatography to purify the protein from cell lysates by standard methods (see, e.g., Ausubel et al., supra). After immuno-affinity chromatography, the protein can be further purified by other standard techniques, e.g., high performance liquid chromatography (see, e.g., Fisher, Laboratory Techniques In Biochemistry And Molecular Biology, Work and Burdon, eds., Elsevier, 1980). In another embodiment, the protein able to specifically block the molecular interaction between p75NTR and PDE4A4/5 is expressed as a fusion protein containing an affinity tag (e.g., GST) that facilitates its purification.

### RNAi

RNA interference (RNAi) can be used to specifically block the molecular interaction between p75NTR and PDE4A4/5. RNAi methods can utilize, for example, small interfering RNAs (siRNA), short hairpin RNA (shRNA), and micro RNAs (miRNA). The following discussion will focus on siRNA, but one skilled in the art will recognize similar approaches are available for other RNAi molecules, such as shRNA and miRNA. The siRNA molecules are produced from long double stranded RNAs (dsRNA) by Dicer, a dsRNA-specific endonuclease, and cause specific degradation of their mRNA-targets by Watson-Crick base-pairing within a multi-enzyme RNA-induced silencing complex (RISC). Design, production, and administration of siRNA molecules as a therapeutic agent is known to the art (see e.g., Pushparaj and Melendez (2006) Clinical and Experimental Pharmacology and Physiology 33(5-6), 504-510; Dillon et al. (2005) Annual Review of Physiology 67, 147-173; Dykxhoorn and Lieberman (2005) Annual Review of Medicine 56, 401-423). RNAi molecules are commercially available from a variety of sources (e.g., Ambion, TX; Sigma Aldrich, MO; Invitrogen).

Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinoformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Administration of siRNA molecules that specific for p75NTR and/or PDE4A4/5 can effect the RNAi-mediated degradation of the target mRNA. For example, a therapeutically effective amount of siRNA specific for p75NTR and/or PDE4A4/5 can be adminstered to patient in need thereof to treat a condition linked to the expression of p75NTR and/or PDE4A4/5.

Generally, an effective amount of siRNA molecule comprises an intercellular concentration at or near the site of misfolding from about 1 nanomolar (nM) to about 100 nM, preferably from about 2 nM to about 50 nM, more preferably from about 2.5 nM to about 10 nM. It is contemplated that greater or lesser amounts of siRNA can be administered.

The siRNA can be administered to the subject by any means suitable for delivering the RNAi molecules to the cells of interest. For example, siRNA molecules can be administered by gene gun, electroporation, or by other suitable parenteral or enteral administration routes, such as intravitreous injection. RNAi molecules can also be administered locally (lung tissue) or systemically (circulatory system) via pulmonary delivery. A variety of pulmonary delivery devices can be effective at delivering Aha1-specific RNAi molecules to a subject (see below). RNAi molecules can be used in conjunction with a variety of delivery and targeting systems, as described in further detail below. For example, siRNA can be encapsulated into targeted polymeric delivery systems designed to promote payload internalization.

The siRNA can be targeted to any stretch of approximately 19-25 contiguous nucleotides in the Aha1 (or other related molecule with similar function) mRNA target sequences. Searches of the human genome database (BLAST) can be carried out to ensure that selected siRNA sequence will not target other gene transcripts. Techniques for selecting target sequences for siRNA are known in the art (see e.g., Reynolds et al. (2004) Nature Biotechnology 22(3), 326-330). Thus, the sense strand of the present siRNA can comprise a nucleotide sequence identical to any contiguous stretch of about 19 to about 25 nucleotides in the target mRNA of p75NTR and PDE4A4/5. Generally, a target sequence on the target mRNA can be selected from a given cDNA sequence corresponding to the target mRNA, preferably beginning 50 to 100 nt downstream (i.e., in the 3′ direction) from the start codon. The target sequence can, however, be located in the 5′ or 3′ untranslated regions, or in the region nearby the start codon.

### Antibodies

Antibodies can be used to specifically block the molecular interaction between p75NTR and PDE4A4/5. For example, antibodies can block the molecular interaction between p75NTR and PDE4A4/5 by specifically binding to p75NTR, PDE4A4/5, and/or the p75NTR-PDE4A4/5 complex. Antibodies within the scope of the invention include, for example, polyclonal antibodies, monoclonal antibodies, antibody fragments, and antibody-based fusion molecules.

Engineering, production, screening, purification, fragmentation, and therapeutic use of antibodies are well known in the art (see generally, Carter (2006) Nat Rev Immunol. 6(5), 343-357; Coligan (2005) Short Protocols in Immunology, John Wiley & Sons, ISBN 0471715786); Teillaud (2005) Expert Opin Biol Ther. 5(Supp. 1) S15-27; Subramanian, ed. (2004) Antibodies: Volume 1: Production and Purification, Springer, ISBN 0306482452; Brent et al., ed. (2003) Current Protocols in Molecular Biology, John Wiley & Sons Inc, ISBN 047150338X; Lo, ed. (2003) Antibody Engineering Methods and Protocols, Humana Press, ISBN 1588290921; Ausubel et al., ed. (2002) Short Protocols in Molecular Biology 5th Ed., Current Protocols, ISBN 0471250929). Various types of antibodies can also be obtained from a variety of commercial sources.

The terminal half-life of antibodies in plasma can be tuned over a wide range, for example several minutes to several weeks, to fit clinical goals for treating conditions linked to the expression of p75NTR and PDE4A4/5 (see e.g., Carter et al. (2006) Nat Rev Immunol. 6(5), 343-357, 353). Chimeric, humanized, and fully human MAbs can effectively overcome potential limitations on the use of antibodies derived from non-human sources to conditions linked to the expression of p75NTR and PDE4A4/5, thus providing decreased immunogenicity with optimized effector functions (see e.g., Teillaud (2005) Expert Opin. Biol. Ther. 5(1), S15-S27; Tomizuka et al. (2000) Proc. Nat. Acad. Sci. USA 97, 722-727; Carter et al. (2006) Nat Rev Immunol. 6(5), 343-357, 346-347). Antibodies can be altered or selected so as to achieve efficient antibody internalization. As such, the antibodies can more effectively interact with target intracellular molecules, such as p75NTR, PDE4A4/5, and/or the p75NTR-PDE4A4/5 complex. Further, antibody-drug conjugates can increase the efficiency of antibody internalization. Efficient antibody internalization can be desirable for delivering specific antibodies to the intracellular environment so as to salvage cAMP levels. Conjugation of antibodies to a variety of agents that can facilitate cellular internalization of antibodies is known in the art (see generally Wu et al. (2005) Nat Biotechnol. 23(9), 1137-1146; McCarron et al. (2005) Mol Intery 5(6), 368-380; Niemeyer (2004) Bioconjugation Protocols, Strategies and Methods, Humana Press, ISBN 1588290980; Hermanson (1996) Bioconjugate Techniques, Academic Press, ISBN 0123423368).

### Small Molecules

Small organic molecules that interact specifically with p75NTR and/or PDE4A4/5 can be used to specifically block the molecular interaction between p75NTR and PDE4A4/5. Identification of a pharmaceutical or small molecule specifically inhibitor of the p75NTR-PDE4A4/5 complex can be readily accomplished through standard high-throughput screening methods. Furthermore, standard medicinal chemistry approaches can be applied to these agents to enhance or modify their activity so as to yield additional agents.

### Aptamers

Purified aptamers that specifically recognize and bind to p75NTR and/or PDE4A4/5 nucleotides or proteins can be used to specifically block the molecular interaction between p75NTR and PDE4A4/5. Aptamers are nucleic acids or peptide molecules selected from a large random sequence pool to bind to specific target molecule. The small size of aptamers makes them easier to synthesize and chemically modify and enables them to access epitopes that otherwise might be blocked or hidden. And aptamers are generally nontoxic and weak antigens because of their close resemblance to endogenous molecules. Generation, selection, and delivery of aptamers is within the skill of the art (see e.g., Lee et al. (2006) Curr Opin Chem Biol. 10, 1-8; Yan et al. (2005) Front Biosci 10, 1802-1827; Hoppe-Seyler and Butz (2000) J Mol Med. 78(8), 426-430). Negative selection procedures can yield aptamers that can finely discriminate between molecular variants. For example, negative selection procedures can yield aptamers that can discriminate between p75NTR, PDE4A4/5 (and/or other phosphodiesterase isoforms), and the p75NTR-PDE4A4/5 binding complex.

Aptamers can also be used to temporally and spatially regulate protein function (e.g., p75NTR and/or PDE4A4/5 function) in cells and organisms. For example, the ligand-regulated peptide (LiRP) system provides a general method where the binding activity of intracellular peptides is controlled by a peptide aptamer in turn regulated by a cell-permeable small molecule (see e.g., Binkowski (2005) Chem & Biol. 12(7), 847-55). Using LiRP or a similar delivery system, the binding activity of p75NTR and/or PDE4A4/5 can be controlled by a cell-permeable small molecule that interacts with the introduced intracellular p75NTR- and/or PDE4A4/5-specific protein aptamer. Thus, aptamers can provide an effective means to modulate the p75NTR-PDE4A4/5 complex activity by, for example, directly binding the p75NTR and/or PDE4A4/5 mRNA, p75NTR and/or PDE4A4/5 expressed protein, and/or the p75NTR-PDE4A4/5 complex.

### Antisense and Ribozyme

Purified antisense nucleic acids that specifically recognize and bind to ribonucleotides encoding p75NTR and/or PDE4A4/5 can be used to block the molecular interaction between p75NTR and PDE4A4/5. Antisense nucleic acid molecules within the invention are those that specifically hybridize (e.g., bind) under cellular conditions to cellular mRNA and/or genomic DNA encoding, for example p75NTR and/or PDE4A4/5 protein, in a manner that inhibits expression of that protein, e.g., by inhibiting transcription and/or translation. Antisense molecules, effective for decreasing p75NTR and/or PDE4A4/5 levels, can be designed, produced, and administered by methods commonly known to the art. (see e.g., Chan et al. (2006) Clinical and Experimental Pharmacology and Physiology 33(5-6), 533-540).

Ribozyme molecules designed to catalytically cleave target mRNA transcripts can also be used to block the molecular interaction between p75NTR and PDE4A4/5. Ribozyme molecules specific for p75NTR and/or PDE4A4/5 can be designed, produced, and administered by methods commonly known to the art (see e.g., Fanning and Symonds (2006) Handbook Experimental Pharmacology 173, 289-303G, reviewing therapeutic use of hammerhead ribozymes and small hairpin RNA). Triplex-forming oligonucleotides can also be used to decrease levels of p75NTR and PDE4A4/5 (see generally, Rogers et al. (2005) Current Medicinal Chemistry 5(4), 319-326).

### Administration

Agents for use in the methods described herein can be delivered in a variety of means known to the art. The agents can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

The agents described herein can be formulated by any conventional manner using one or more pharmaceutically acceptable carriers and/or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of the agent, preferably in purified form, together with a suitable amount of carrier so as to provide the form for proper administration to the subject. The formulation should suit the mode of administration. The agents of use with the current invention can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents of the present invention and/or together with other biologically active or biologically inert agents. Such biologically active or inert agents may be in fluid or mechanical communication with the agent(s) or attached to the agent(s) by ionic, covalent, Van der Weals, hydrophobic, hydrophillic or other physical forces.

When used in the methods of the invention, a therapeutically effective amount of one of the agents described herein can be employed in pure form or, where such forms exist, in pharmaceutically acceptable salt form and with or without a pharmaceutically acceptable excipient. For example, the agents of the invention can be administered, at a reasonable benefit/risk ratio applicable to any medical treatment, in a sufficient amount sufficient to inhibit and/or relieve symptoms associated with p75NTR and/or PDE4A4/5 expression. Administration of an effective amount of an agent that disrupts the molecular interaction of p75NTR and PDE4A4/5 can increase tPA activity, decrease fibrin levels, increase finbrin degradation, increase extracellular proteolysis, decrease degradation of cAMP by phosphodiesterase, and/or increase PKA activity.

Toxicity and therapeutic efficacy of such agents can be determined by standard pharmaceutical procedures in cell cultures and/or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where large therapeutic indices are preferred.

The amount of an agent that may be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses. Agent administration can occur as a single event or over a time course of treatment. For example, an agent can be administered daily, weekly, bi-weekly, or monthly. For some conditions, treatment could extend from several weeks to several months or even a year or more.

The specific therapeutically effective dose level for any particular subject will depend upon a variety of factors including the condition being treated and the severity of the condition; activity of the specific agent employed; the specific composition employed; the age, body weight, general health, sex and diet of the patient; the time of administration; the route of administration; the rate of excretion of the specific agent employed; the duration of the treatment; drugs used in combination or coincidental with the specific agent employed and like factors well known in the medical arts. It will be understood by a skilled practitioner that the total daily usage of the agents for use in the present invention will be decided by the attending physician within the scope of sound medical judgment.

Agents that block the molecular interaction between p75NTR and PDE4A4/5 can also be used in combination with other therapeutic modalities. Thus, in addition to the therapies described herein, one may also provide to the subject other therapies known to be efficacious for particular conditions linked to p75NTR and PDE4A4/5 expression.

Controlled-release (or sustained-release) preparations may be formulated to extend the activity of the agent and reduce dosage frequency. Controlled-release preparations can also be used to effect the time of onset of action or other characteristics, such as blood levels of the agent, and consequently affect the occurrence of side effects.

Controlled-release preparations may be designed to initially release an amount of an agent that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized and/or excreted from the body. The controlled-release of an agent may be stimulated by various inducers, e.g., change in pH, change in temperature, enzymes, water, or other physiological conditions or molecules.

Controlled-release systems may include, for example, an infusion pump which may be used to administer the agent in a manner similar to that used for delivering insulin or chemotherapy to specific organs or tumors. Typically, using such a system, the agent is administered in combination with a biodegradable, biocompatible polymeric implant (see below) that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

The agents of the invention may be administered by other controlled-release means or delivery devices that are well known to those of ordinary skill in the art. These include, for example, hydropropylmethyl cellulose, other polymer matrices, gels, permeable membranes, osmotic systems, multilayer coatings, microparticles, liposomes, microspheres, or the like, or a combination of any of the above to provide the desired release profile in varying proportions (see below). Other methods of controlled-release delivery of agents will be known to the skilled artisan and are within the scope of the invention.

Agents that block the molecular interaction between p75NTR and PDE4A4/5 can be administered through a variety of routes well known in the arts. Examples include methods involving direct injection (e.g., systemic or stereotactic), implantation of cells engineered to secrete the factor of interest, drug-releasing biomaterials, implantable matrix devices, implantable pumps, injectable gels and hydrogels, liposomes, micelles (e.g., up to 30 μm), nanospheres (e.g., less than 1 μm), microspheres (e.g., 1-100 μm), reservoir devices, etc.

Pulmonary delivery of macromoles and/or drugs, such as the agents described herein, provide for relatively easy, non-invasive administration to the local tissue of the lungs or the circulatory system for systemic circulation (see e.g., Cryan (2004) AAPS J. 7(1) article 4, E20-41, providing a review of pulmonary delivery technology). Advantages of pulmonary delivery include noninvasiveness, large surface area for absorption (˜75 m2), thin (˜0.1 to 0.5 μm) alveolar epitheliuem permitting rapid absorption, absence of first pass metabolism, decreased proteolytic activity, rapid onset of action, and high bioavailablity. Drug formulations for pulmonary delivery, with or without excipients and/or a dispersible liquid, are known to the art. Carrier-based systems for biomolecule delivery, such as polymeric delivery systems, liposomes, and micronized carbohydrates, can be used in conjunction with pulmonary delivery. Penetration enhancers (e.g., surfactants, bile salts, cyclodextrins, enzyme inhibitors (e.g., chymostatin, leupeptin, bacitracin), and carriers (e.g., microspheres and liposomes) can be used to enhance uptake across the alveolar epithelial cells for systemic distribution. Various inhalation delivery devices, such as metered-dose inhalers, nebulizers, and dry-powder inhalers, that can be used to deliver the biomolecules described herein are known to the art (e.g., AErx (Aradigm, Calif.); Respimat (Boehringer, Germany); AeroDose (Aerogen Inc., CA)). As known in the art, device selection can depend upon the state of the biomolecule (e.g., solution or dry powder) to be used, the method and state of storage, the choice of excipients, and the interactions between the formulation and the device. Dry powder inhalation devices are particularly preferred for pulmonary delivery of protein-based agents (e.g., Spinhaler (Fisons Pharmaceuticals, NY); Rotohaler (GSK, NC); Diskhaler (GSK, NC); Spiros (Dura Pharmaceuticals, CA); Nektar (Nektar Pharmaceuticals, CA)). Dry powder formulation of the active biological ingredient to provide good flow, dispersability, and stability is known to those skilled in the art.

Agents that block the molecular interaction between p75NTR and PDE4A4/5 can be encapsulated and administered in a variety of carrier delivery systems. Examples of carrier delivery systems include microspheres, hydrogels, polymeric implants, smart ploymeric carriers, and liposomes. Carrier-based systems for biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; and/or improve shelf life of the product.

Polymeric microspheres can be produced using naturally occurring or synthetic polymers and are particulate systems in the size range of 0.1 to 500 μm. Polymeric micelles and polymeromes are polymeric delivery vehicles with similar characteristics to microspheres and can also facilitate encapsulation and delivery of the biomolecules described herein. Fabrication, encapsulation, and stabilization of microspheres for a variety of biomolecule payloads are within the skill of the art (see e.g., Varde & Pack (2004) Expert Opin. Biol. 4(1) 35-51). Release rate of microspheres can be tailored by type of polymer, polymer molecular weight, copolymer composition, excipients added to the microsphere formulation, and microsphere size. Polymer materials useful for forming microspheres include PLA, PLGA, PLGA coated with DPPC, DPPC, DSPC, EVAc, gelatin, albumin, chitosan, dextran, DL-PLG, SDLMs, PEG (e.g., ProMaxx), sodium hyaluronate, diketopiperazine derivatives (e.g., Technosphere), calcium phosphate-PEG particles, and oligosaccharide derivative DPPG (e.g., Solidose). Encapsulation can be accomplished, for example, using a water/oil single emulsion method, a water-oil-water double emulsion method, or lyophilization. Several commercial encapsulation technologies are available (e.g., ProLease®, Alkerme). Microspheres encapsulating the agents described herein can be administered in a variety of means including parenteral, oral, pulmonary, implantation, and pumping device.

Polymeric hydrogels, composed of hydrophillic polymers such as collagen, fibrin, and alginate, can also be used for the sustained release of agents that decrease levels of Aha1 and/or other related molecules with similar function (see generally, Sakiyama et al. (2001) FASEB J. 15, 1300-1302).

Three-dimensional polymeric implants, on the millimeter to centimeter scale, can be loaded with agents that decrease levels of Aha1 and/or other related molecules with similar function (see generally, Teng et al (2002) Proc. Natl. Acad. Sci. U.S.A. 99, 3024-3029). A polymeric implant typically provides a larger depot of the bioactive factor. The implants can also be fabricated into structural supports, tailoring the geometry (e.g., shape, size, porosity) to the application. Implantable matrix-based delivery systems are also commercially available in a variety of sizes and delivery profiles (e.g., Innovative Research of America, Sarasota, Fla.).

“Smart” polymeric carriers can be used to administer agents that block the molecular interaction between p75NTR and PDE4A4/5 (see generally, Stayton et al. (2005) Orthod Craniofacial Res 8, 219-225; Wu et al. (2005) Nature Biotech (2005) 23(9), 1137-1146). Carriers of this type utilize polymers that are hydrophilic and stealth-like at physiological pH, but become hydrophobic and membrane-destabilizing after uptake into the endosomal compartment (i.e., acidic stimuli from endosomal pH gradient) where they enhance the release of the cargo molecule into the cytoplasm. Design of the smart polymeric carrier can incorporate pH-sensing functionalities, hydrophobic membrane-destabilizing groups, versatile conjugation and/or complexation elements to allow the drug incorporation, and an optional cell targeting component. Potential therapeutic macromolecular cargo includes peptides, proteins, antibodies, polynucleotides, plasmid DNA (pDNA), aptamers, antisense oligodeoxynucleotides, silencing RNA, and/or ribozymes that effect a decrease in levels of Aha1 and/or related molecules with similar function. As an example, smart polymeric carriers, internalized through receptor mediated endocytosis, can enhance the cytoplasmic delivery of Aha1-targeted siRNA, and/or other agents described herein. Polymeric carriers include, for example, the family of poly(alkylacrylic acid) polymers, specific examples including poly(methylacrylic acid), poly(ethylacrylic acid) (PEAA), poly(propylacrylic acid) (PPAA), and poly(butylacrylic acid) (PBAA), where the alkyl group progressively increased by one methylene group. Smart polymeric carriers with potent pH-responsive, membrane destabilizing activity can be designed to be below the renal excretion size limit. For example, poly(EAA-co-BA-co-PDSA) and poly(PAA-co-BA-co-PDSA) polymers exhibit high hemolytic/membrane destabilizing activity at the low molecular weights of 9 and 12 kDa, respectively. Various linker chemistries are available to provide degradable conjugation sites for proteins, nucleic acids, and/or targeting moieties. For example, pyridyl disulfide acrylate (PDSA) monomer allow efficient conjugation reactions through disulfide linkages that can be reduced in the cytoplasm after endosomal translocation of the therapeutics.

Liposomes can be used to administer agents that block the molecular interaction between p75NTR and PDE4A4/5. The drug carrying capacity and release rate of liposomes can depend on the lipid composition, size, charge, drug/lipid ratio, and method of delivery. Conventional liposomes are composed of neutral or anionic lipids (natural or synthetic). Commonly used lipids are lecithins such as (phosphatidylcholines), phosphatidylethanolamines (PE), sphingomyelins, phosphatidylserines, phosphatidylglycerols (PG), and phosphatidylinositols (PI). Liposome encapsulation methods are commonly known in the arts (Galovic et al. (2002) Eur. J. Pharm. Sci. 15, 441-448; Wagner et al. (2002) J. Liposome Res. 12, 259-270). Targeted liposomes and reactive liposomes can also be used to deliver the biomolecules of the invention. Targeted liposomes have targeting ligands, such as monoclonal antibodies or lectins, attached to their surface, allowing interaction with specific receptors and/or cell types. Reactive or polymorphic liposomes include a wide range of liposomes, the common property of which is their tendency to change their phase and structure upon a particular interaction (eg, pH-sensitive liposomes) (see e.g., Lasic (1997) Liposomes in Gene Delivery, CRC Press, FL).

Various other delivery systems are known in the art and can be used to administer the agents of the invention. Moreover, these and other delivery systems may be combined and/or modified to optimize the administration of the agents of the present invention.

Screening

Another aspect of the invention is directed to a system for screening candidate agents for actions on p75NTR, PDE4A4/5 and/or the p75NTR-PDE4A4/5 complex. In one embodiment, candidate agents are screened for the ability to specifically block molecular interaction between p75NTR and PDE4A4/5, which can be useful for the development of compositions for therapeutic or prophylactic treatment of conditions associated with p75NTR and PDE4A4/5 expression. Assays can be performed on living mammalian cells, which more closely approximate the effects of a particular serum level of drug in the body. Alternatively, assays can be performed with isolated p75NTR and PDE4A4/5 in vitro. Cell lines expressing increased or decreased amounts of p75NTR and/or PDE4A4/5 protein would be useful for evaluating the activity of potential bioactive agents, or on extracts prepared from the cultured cell lines. Studies using extracts offer the possibility of a more rigorous determination of direct agent/enzyme interactions.

Thus, the present invention may provide a method to evaluate an agent to specifically block molecular interaction between p75NTR and PDE4A4/5, and thus to prevent elevated cAMP degradation in a mammalian host, preferably a human host. Candidate agents can include, but are not limited to, nucleic acids, polypeptides, siRNAs, antisense molecules, aptamers, ribozymes, triple helices, antibodies, and small inorganic molecules. The assay may comprise contacting the a transgenic cell line or an extract thereof with a preselected amount of the agent in a suitable culture medium or buffer, and measuring the level of activity of the p75NTR-PDE4A4/5 complex, as compared to a control cell line or portion of extract in the absence of said agent and/or a control cell line expressing altered levels of p75NTR and/or PDE4A4/5 protein. Alternatively, the assay may comprise contacting p75NTR and PDE4A4/5 with a preselected amount of the agent in a suitable medium or buffer, and measuring the level of activity of the p75NTR-PDE4A4/5 complex, as compared to a control in the absence of said agent and/or a control with differing levels of p75NTR and/or PDE4A4/5 protein.

More specifically, a candidate agent for the treatment of a condition linked to p75NTR and/or PDE4A4/5 can be screened by providing a cell stably expressing both proteins in a suitable culture medium or buffer, administering the candidate agent to the cell, measuring the activity levels of p75NTR-PDE4A4/5 complex in the cell, and determining whether the candidate agent decreases intracellular p75NTR-PDE4A4/5 complex activity level. Alternatively, the assay may be conducted in vitro with isolated p75NTR and/or PDE4A4/5. Desirable candidates will generally possess the ability to block molecular interaction between p75NTR and PDE4A4/5. Preferably, such desirable candidates will specifically block molecular interaction between p75NTR and PDE4A4/5. Also preferably, identified agents do not substantially interfere with other phosphodiesterase isoforms.

Any method suitable for detecting levels of p75NTR, PDE4A4/5, and/or p75NTR-PDE4A4/5 complex may be employed for determining levels resultant from administration of the candidate agent. Among the traditional methods which may be employed are co-immunoprecipitation, crosslinking, co-purification through gradients or chromatographic columns, and activity assays. Utilizing procedures such as these allows for the identification of the proteins and/or complexes of interest.

The present invention also comprises the use of p75NTR and PDE4A4/5 in drug discovery efforts to elucidate relationships that exist between these proteins and a disease state, phenotype, or condition. These methods include detecting or decreasing levels of p75NTR-PDE4A4/5 complex comprising contacting a sample, tissue, cell, or organism with the agents of the present invention, measuring the activity of the p75NTR-PDE4A4/5 complex, and/or a related phenotypic or chemical endpoint at some time after treatment, and optionally comparing the measured value to a non-treated sample or sample treated with a further agent of the invention. These methods can also be performed in parallel or in combination with other experiments to determine the function of unknown genes for the process of target validation or to determine the validity of a particular gene product as a target for treatment or prevention of a particular disease, condition, or phenotype.

### Discussion

The studies reported herein identify p75NTR as a novel player that regulates proteolyticactivity and fibrin degradation during peripheral nerve regeneration and pulmonaryfibrosis via directly binding to phosphodiesterases and decreasing intracellular cAMP. These data show for the first time that a) plasminogen activation is down regulated by a neurotrophin receptor via a cAMP/PKA mechanism, b) p75NTR induces degradation of cAMP and c) phosphodiesterases can be recruited to the membrane via direct binding to a transmembrane receptor.

Without being bound by a particular theory, it is possible that p75NTR plays the following role in the regulation of plasminogen activation (see e.g., FIG. 10). Injury induces upregulation of p75NTR in a variety of cell types within and outside of the nervous system. p75NTR directly interacts with PDE4A5 and induces degradation of cAMP resulting in decreased PKA activity. Downregulation of cAMP induces upregulation of PAI-1 and downregulation of tPA resulting in decreased extracellular proteolysis. And decreased proteolytic activity inhibits extracellular matrix remodeling and fibrinolysis in the sciatic nerve and the lung. Given the effects of the tPA/plasmin system in the regulation of cell migration in cerebellar granule neurons (Seeds et al., 1999) and cell death in the hippocampus (Chen and Strickland, 1997; Tsirka et al., 1995) and secretion of growth factors, such as TGF-β (Odekon et al., 1994), p75NTR may be upstream of other cellular functions associated with the proteolytic system (see e.g., FIG. 10). Another substrate of plasmin are proneurotrophins, the high affinity ligands of p75NTR (Lee et al., 2001). Cleavage of pro-BDNF by tPA/plasmin system was recently implicated in LTP (Pang et al., 2004). Inhibition of plasmin activation by p75NTR may regulate the balance between neurotrophins and their precursors and favor the accumulation of proneurotrophins. In addition, given the multiple genes regulated by cAMP, other cellular functions may be regulated by p75NTR/cAMP signaling (see e.g., FIG. 10).

Also, increased expression of p75NTR by neurons, glia and brain endothelial cells may regulate the temporal and spatial pattern of tPA expression during brain injury or inflammation. Given the dependence of p75NTR functions on the availability of different ligands and co-receptors (Teng and Hempstead, 2004), p75NTR may contribute in plasminogen activation and ECM remodeling in different injury models. Data disclosed herein indicates that expression of p75NTR can inhibit tPA in the absence of neurotrophin ligands and/or in the absence of serum. It has been previously shown that p75NTR may signal in a ligand-independent manner to induce neuronal apoptosis (Rabizadeh et al., 1993). Thus, non-neurotrophin ligands that bind directly to p75NTR, such as β-amyloid (Year et al., 2002) and prion peptides (Della-Bianca et al., 2001), as well as Nogo, MAG and OMgp, NogoR/p75NTR-dependent inhibitors of nerve regeneration (Filbin, 2003), may be involved in the regulation of plasminogen activation in neuronal cells.

In addition to fibrinolysis, the tPA/plasmin proteolytic system is also involved in neurite outgrowth and pathfinding, memory formation, emotion and neurodegeneration (Madani et al., 2003). tPA can cleave and potentiate the signaling of the N-methyl-Daspartate (NMDA) receptor resulting in increased neuronal Ca++ influx (Nicole et al., 2001). This mechanism has been proposed (Benchenane et al., 2004) as a regulatory mechanism for tPA-mediated neuronal death, long term potentiation (LTP) (Baranes et al., 1998) and cerebellar motor learning (Seeds et al., 2003). Overall, given the wide range of the tPA/plasmin substrates, p75NTR and the tPA/plasmin system may regulate many functions, such as neuronal survival, plasticity, and death during development or after injury.

Regulation of cAMP is a novel signaling mechanism downstream of p75NTR, which, by recruiting PDE4A5, targets cAMP degradation and decreases PKA activity. The direct interaction between p75NTR and PDE4A5 represents the first example of recruitment of PDEs to the membrane by direct binding to a transmembrane receptor. Compartmentalization of PDEs represents a major mechanism that regulates intracellular specificity of cAMP signaling (Brunton, 2003). It has been previously shown that β-arrestin binding to the N-terminal regions of PDE4s (Bolger et al., 2003) targets degradation of cAMP to the membrane (Perry et al., 2002). As shown herein, p75NTR utilizes three binding motifs on PDE4A5, namely within the LR1, catalytic, and C-terminal subunits to mediate its recruitment to the membrane. LR1 domain is unique for the PDE4A subfamily, while the C-terminal domain is unique for each PDE4 subfamily. The extreme C-terminus of PDE4A5 is shown to be the major interacting domain with p75NTR; thus providing the first evidence for a role of the C-terminal domain as a regulator of isoform-specific phosphodieterase recruitment to subcellular locations. Recent evidence has described important biological functions for PDE4D in ischemic stroke (Gretarsdottir et al., 2003) and heart failure (Lehnart et al., 2005) and for PDE4B in schizophrenia (Millar et al., 2005). Shown herein is the biological function for PDE4A5 as a molecular mediator of p75NTR/cAMP signaling that regulates plasminogen activation and fibrinolysis.

PDE4 is expressed both in the lung (Richter et al., 2005) and in neural tissues (Jin et al., 1999). In the lung, specific PDE4 inhibitors have been used for the clinical treatment of respiratory diseases (Spina, 2003). In spinal cord injury in rodents, elevation of cAMP via specific inhibition of PDE4 by rolipram promotes axonal regeneration and functional recovery (Nikulina et al., 2004; Pearse et al., 2004). In the sciatic nerve, reduction of cAMP after crush or permanent transection is attributed primarily to upregulation of PDE4 by SCs, the cells that upregulate p75NTR after nerve injury (Walikonis and Poduslo, 1998). Based on results disclosed herein, both in the lung and the nervous system, re-expression of p75NTR after injury may contribute to the activation of PDE4. In corticospinal tract axons, cAMP controls the ability of neurons to regenerate (Cai et al., 2001; Cai et al., 1999) and elevation of cAMP via inhibition of PDE4 overcomes the inhibition of neuronal regeneration by myelin (Gao et al., 2003). It has been reported that neurotrophin signaling via Trk receptors elevates cAMP and overcomes the inhibition of nerve regeneration by myelin proteins via inhibition of PDE4 (Gao et al., 2003). p75NTR may exert the opposite function as Trk receptors by recruiting PDE4A5. PDE4A has been detected as the predominant PDE4 isoform at the corticospinal tract (Chemy and Davis, 1999). Because p75NTR can act as a co-receptor for NogoR, a mediator of the inhibition of nerve regeneration, PDE4A activation by p75NTR may play an inhibitory role in nerve regeneration by competing with neurotrophin signaling via Trk receptors.

Also, p75NTR may play a role as a regulator of fibrin deposition in the lung. While not ebing bound by any particular theory, a suggested mechanism for the function of p75NTR in the lung is that NGF/p75NTR signaling may enhance local neurogenic inflammation leading to exacerbated pulmonary disease (Renz et al., 2004). The studies herein support an additional pathway for the damaging role of p75NTR in the lung as a regulator of expression of PAI-1 and a mediator of fibrosis. Expression of p75NTR in the lung is detected mainly in sympathetic neurons and basal epithelial cells of bronchioles (Mark Bothwell, personal communication). Similar to p75NTR, PAI-1 is expressed by bronchial epithelial cells after LPS stimulation (Savoy et al., 2003) and its expression is considered to result in an antifibrinolytic environment within the airway wall. Expression of p75NTR can increase PAI-1 expression in the bronchial epithelium and therefore increase subepithelial fibrin deposition. Several mechanisms have been proposed for the participation of fibrin in lung pathogenesis, including regulation of the inflammatory response and airway remodeling (Idell, 2003; Savoy et al., 2003). Thus, p75NTR-mediated regulation of PAI-1 may influence inflammatory and tissue repair processes in pulmonary disease.

Taken together, these data identify a novel, cAMP-dependent signaling pathway initiated by p75NTR that regulates plasminogen activation and perpetuation of scar formation after sciatic nerve and lung injury. The p75NTR, the first member of the TNF receptor superfamily, modulates a variety of cell survival and death decisions (Chao, 2003). The novel signaling pathway downstream of p75NTR, identified herein, directly links p75NTR to phosphodiesterase-mediated degradation of cAMP. Provided herein is a novel perspective for the role of the p75NTR upregulation at sites of injury as a regulator of ECM remodeling by suppressing activation of plasminogen. The association of p75NTR with inhibition of extracellular proteolysis supports a novel mechanism for p75NTR-mediated regulation of disease progression via accumulation of plasmin-cleaved substrates in both neuronal and non-neuronal tissues.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Fibrin Deposition is Reduced in p75NTR−/− Mice

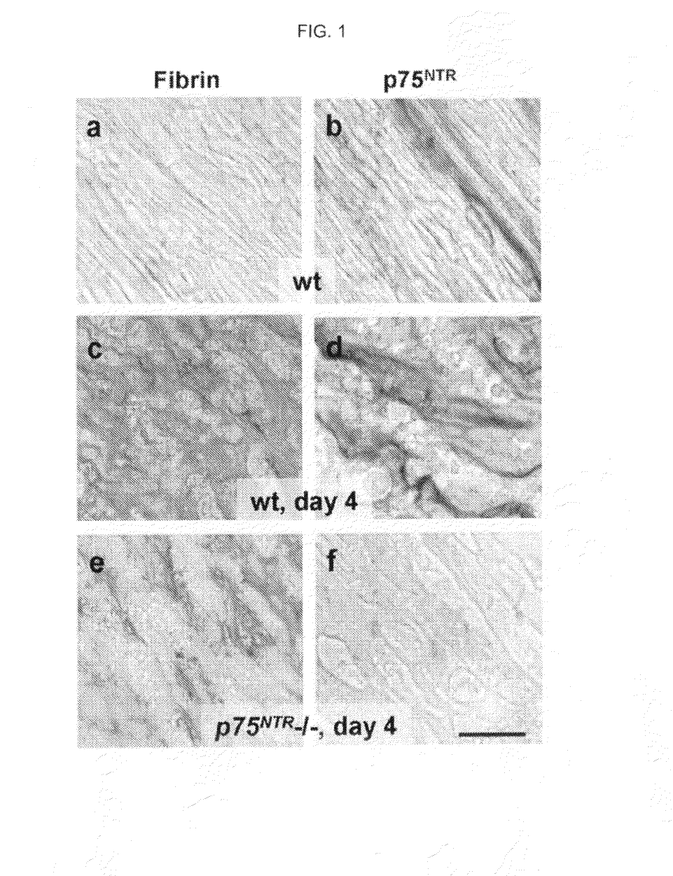

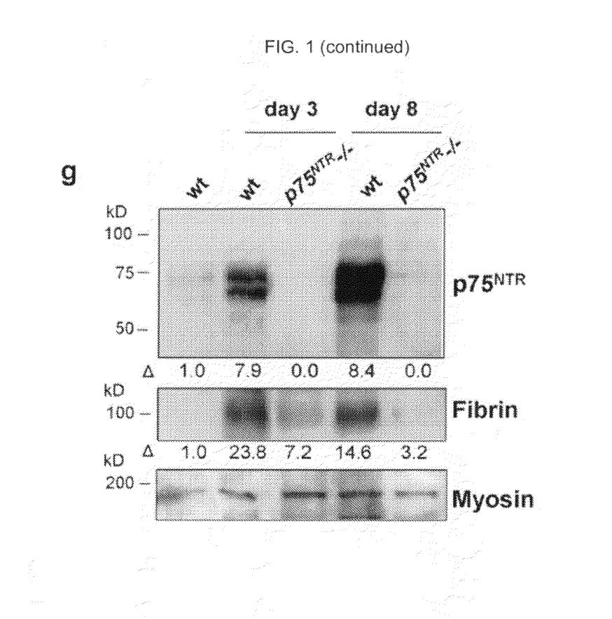

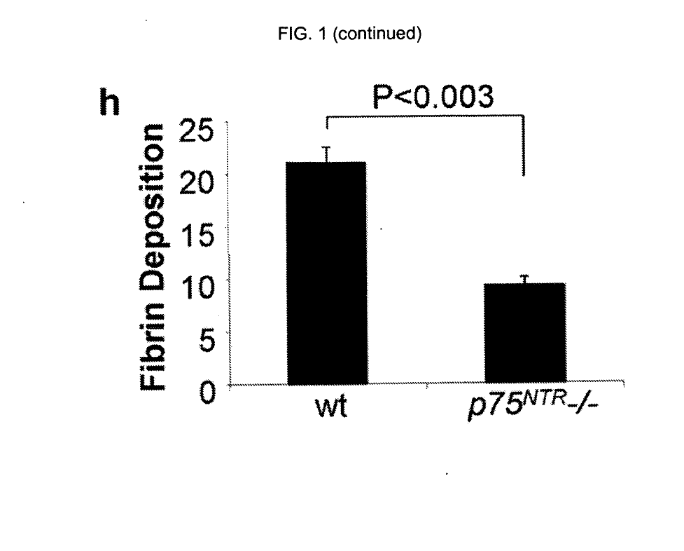

To examine whether p75NTR regulates fibrin deposition in the sciatic nerve fibrin levels in wild-type (wt) and p75NTR−/− mice were compared after injury. FIG. 1 depicts immunohistochemistry for fibrin on uninjured wt (a) and 4 d after sciatic nerve crush injury wt (c) and p75NTR−/− mice (e). Immunohistochemistry for p75NTR on uninjured wt (b) and 4 d after sciatic nerve crush injury wt (d) and p75NTR−/− mice (f). Representative images are shown from n=20 wt and n=20 p75NTR−/− mice. (g) Western blot for p75NTR and fibrin on sciatic nerve extracts from uninjured wt, and wt and p75NTR−/− mice 3 and 8 d after injury. Myosin serves as loading control. Western blots were performed three times. A representative blot is shown. (h) Quantification of fibrin deposition shows significant decrease for fibrin in p75NTR−/− mice (n=5), when compared with wt mice (n=4). Bar graph represents means±SEM (P<0.003; by t test). Bar, 25 μm.

In wt mice, there is a dramatic increase of fibrin deposition (FIG. 1c) and p75NTR expression (FIG. 1d) after injury, when compared with uninjured nerves (FIG. 1, a and b). In contrast, p75NTR−/− mice show reduced fibrin deposition after injury (FIG. 1 e). Quantification of immunoblots reveals that p75NTR−/− mice have decreased fibrin by threefold 3 d and fourfold 8 d after injury (FIG. 1 g). Quantification of fibrin immunostaining also reveals that p75NTR−/− mice have significantly decreased fibrin (FIG. 1 h, P<0.003). These results suggest that loss of p75NTR decreases the levels of fibrin in the sciatic nerve after injury.

### Example 2

### p75NTR Regulates Expression of tPA in the Sciatic Nerve after Crush Injury

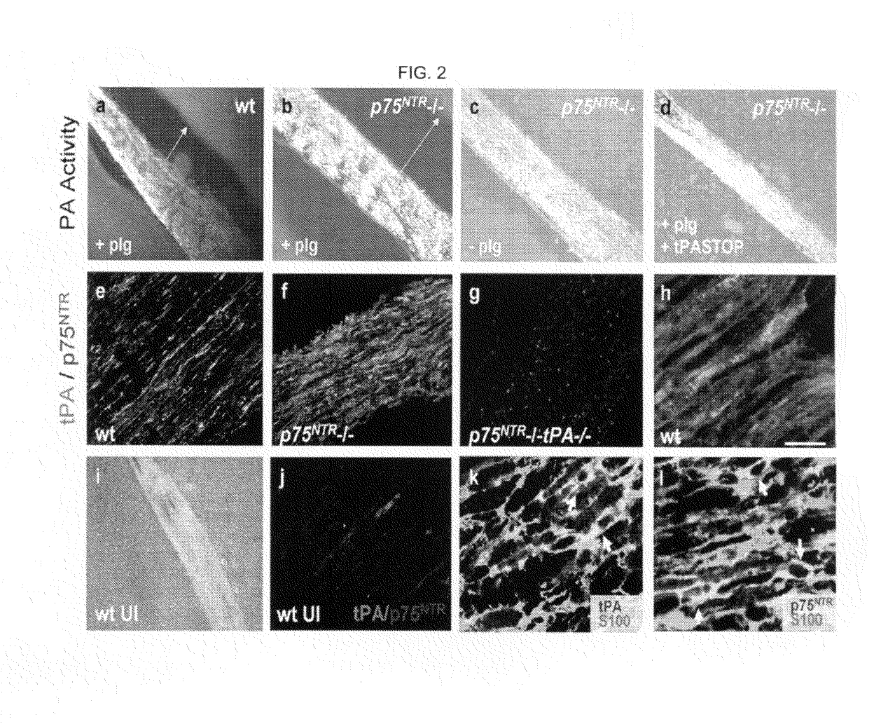

Analysis of total fibrinogen levels were similar in the plasma of wt and p75NTR−/− mice (unpublished data), suggesting the decrease in fibrin deposition is not the result of hypofibrinogenemia. FIG. 2 depicts in situ zymography in the presence of plasminogen on wt (a) and p75NTR−/− (b) mice and in the absence of plasminogen (c) or in the presence of plasminogen and tPASTOP (d) in p75NTR−/− mice. Arrows indicate the lytic zone. Double immunofluorescence for tPA (green) or p75NTR (red) on wt (e and h), p75NTR−/− (f) and p75NTR−/−tPA−/− mice (g). Uninjured wt sciatic nerve exhibits minimal proteolytic activity (i) and minimal tPA and p75NTR immunoreactivity (j). Zymographies have been performed on n=10 wt and n=10 p75NTR−/− mice. Representative images are shown. tPA (k) and p75NTR (l) expression in SCs was verified by double immunofluorescence with an S100 (SC marker) antibody. Arrows indicate double-positive cells (k and l, yellow). The experiment was repeated at two different time points (4 and 8 d after crush injury) in n=4 mice per genotype per time point and representative images are shown. Bar: 400 μm (a-d, i), 150 μm (e-g, j), 20 μm (h, k, and l).

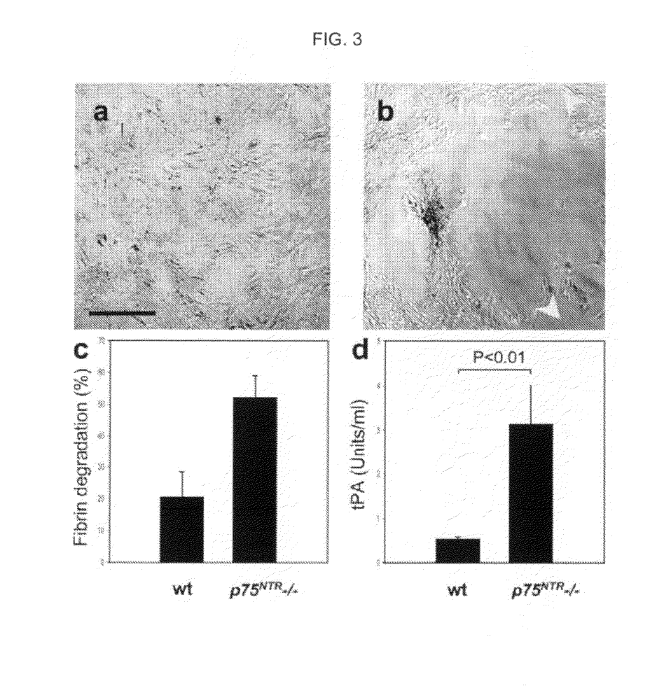

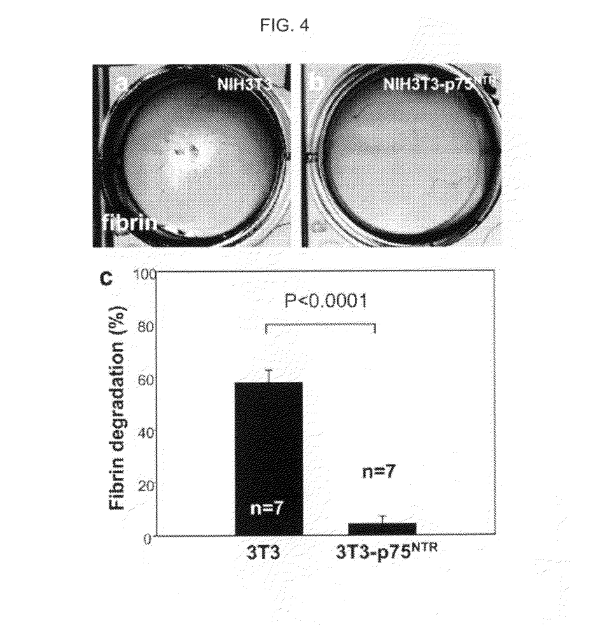

Fibrin removal depends on proteolytic activity (Bugge et al., 1996), providing that the decreased fibrin in the p75NTR−/− mice reflects an up-regulation of the proteolytic activity. p75NTR−/− mice have increased proteolytic activity (FIG. 2 b) when compared with wt mice (FIG. 2 a) that is statistically significant (FIG. 3 i, P<0.05). Uninjured nerves exhibit minimal proteolytic activity (FIG. 2 i), as expected (Akassoglou et al., 2000). Injured p75NTR−/− sciatic nerves do not show lysis of fibrin in the absence of plasminogen (FIG. 2 c), suggesting that the proteolytic activity is plasminogen dependent. The tPA/plasmin system regulates fibrin clearance after nerve injury (Akassoglou et al., 2000). A specific tPA inhibitor, tPASTOP, blocks proteolytic activity in p75NTR−/− mice (FIG. 2 d). p75NTR is strongly activated by withdrawal of axons (Lemke and Chao, 1988) and its expression correlates with proliferating, non-myelin producing Schwann cells (SCs) (Zorick and Lemke, 1996). After sciatic nerve injury both p75NTR (FIG. 2 e, red) and tPA (FIG. 2 e, arrows) increase when compared with uninjured controls (FIG. 2 j), but show little colocalization (FIG. 2, e and h), suggesting that p75NTR-reexpressing SCs do not express tPA. Expression of tPA (FIG. 2 k, red) and p75NTR (FIG. 2 l, arrows) in SCs is confirmed using double immunofluorescence with the SC marker S100 (FIG. 2, k and l; arrows).

### Example 3

### Genetic Loss of tPA Rescues the Effects of p75NTR Deficiency

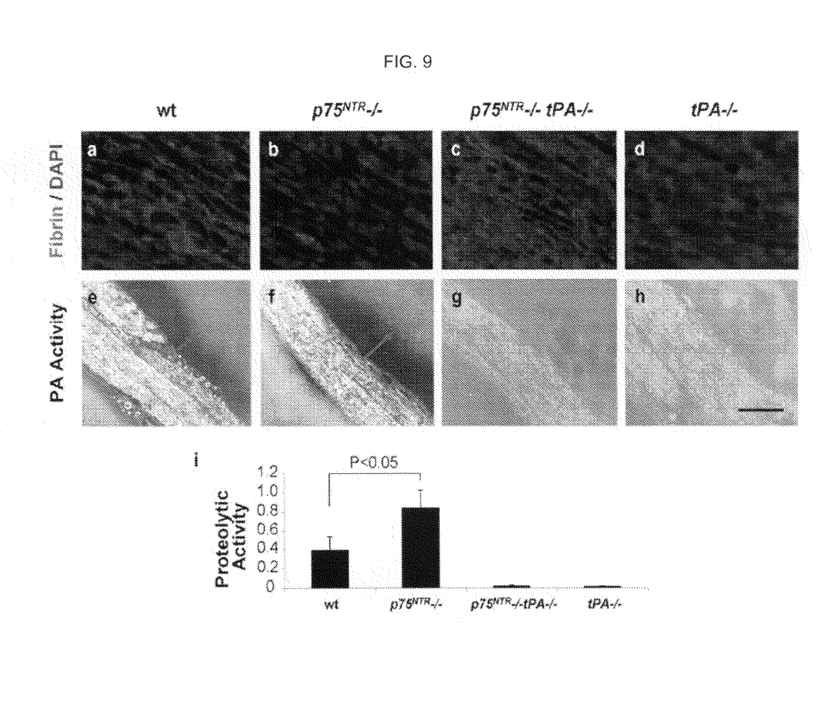

To examine genetically whether the increased proteolytic activity in the p75NTR−/− mice was due to tPA, we crossed p75NTR−/− mice with tPA−/− mice and generated p75NTR−/−tPA−/− doubleknockout mice. FIG. 9 depicts increased fibrin deposition in the crushed sciatic nerve of p75NTR−−tPA−/− mice (c), when compared with crushed p75NTR−/− sciatic nerve (b). Wt (a) and tPA−/− (d) nerves are used for control. In situ zymography shows lack of proteolytic activity in the crushed p75NTR−/−tPA−/− sciatic nerves (n=5) (g), when compared with crushed p75NTR−/− sciatic nerves (n=20) (f). Crushed wt (e) and tPA−/− (h) nerves are used for control. Fibrin immunostainings and zymographies were performed on n=5 p75NTR−−tPA−/−, n=20 p75NTR−/−, n=20 wt, n=5 tPA−/− mice. Representative images are shown. (i) Quantification of proteolytic activity 4 d after crush injury shows statistically significant increase for proteolytic activity in p75NTR−/− mice. Quantification results are based on n=5 p75NTR−/−, n=5 p75NTR−/− tPA−/−, n=5 tPA−/− and n=4 wt mice. Bar graph represents means±SEM (*, P<0.05; by ANOVA). Bar: 50 μm (a-d), 300 μm (e-h).

p75NTR−/− mice show a decrease in fibrin deposition (FIG. 9 b) and an increase in proteolytic activity (FIG. 9 f), compared with wt control mice (FIG. 9, a and e, respectively). In contrast, p75NTR−/−tPA−/− mice show increased fi brin deposition (FIG. 9 c) when compared with p75NTR−/− mice (FIG. 9 b) and no evidence of proteolytic activity (FIG. 9 g). As a control, tPA−/− mice also show no evidence of proteolytic activity after sciatic nerve crush injury (FIG. 9 h), as described previously (Akassoglou et al., 2000). Quantification of proteolytic activity is shown in FIG. 9 i. The evidence derived from the genetic depletion of tPA in the p75NTR−/− mice (p75NTR−/−tPA−/− mice, FIG. 9 g) are in accordance with the pharmacologic inhibition of tPA activity in the p75NTR−/− sciatic nerve using tPASTOP (FIG. 2 d). Overall, these results suggest that up-regulation of proteolytic activity in the sciatic nerve of p75NTR−/− mice is due to upregulation of tPA.

### Example 4

### p75NTR−/− Schwann Cells Show Increased Expression of tPA and Increased Fibrinolysis

Because SCs are a major source for tPA after injury, primary SCs were isolated from wt and p75NTR−/− mice and cultured them on a three-dimensional (3D) fibrin gel. FIG. 3 depicts primary SC cultures from wt (a) or p75NTR−/− mice (b) on a 3D fibrin gel. Arrowheads indicate the border of fibrin degradation. Quantification of fibrin degradation (c) and tPA activity (d) from wt and p75NTR−/− SCs. Experiments were performed three times in duplicates. Representative images are shown. Bar graph represents means±SEM (P<0.01; by t test). Bar, 130 μm.

Wt SCs, which express high levels of p75NTR, form a monolayer on the fibrin gel (FIG. 3 a). In contrast, p75NTR−/− SCs degrade the fibrin gel (FIG. 3 b) and show a 2.7-fold increase of fibrin degradation (FIG. 3 c). p75NTR−/− SCs show a sixfold increase in tPA levels, when compared with wt SCs (FIG. 3 d; P<0.01). These results suggest that p75NTR down-regulates tPA activity and blocks fibrin degradation in SCs in vitro.

### Example 5

### Expression of p75NTR Inhibits tPA and Fibrinolysis

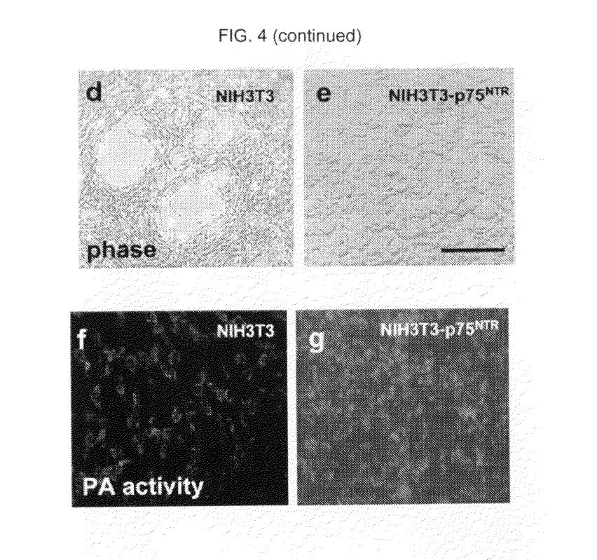

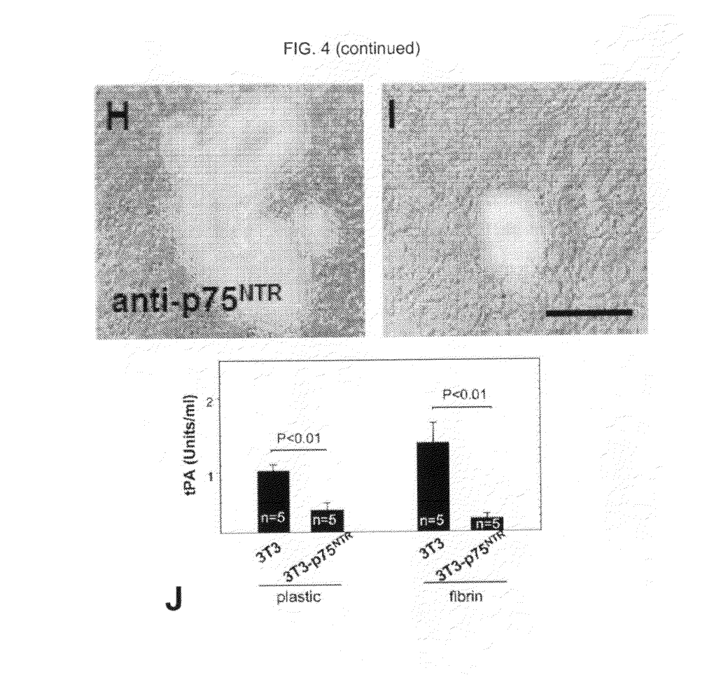

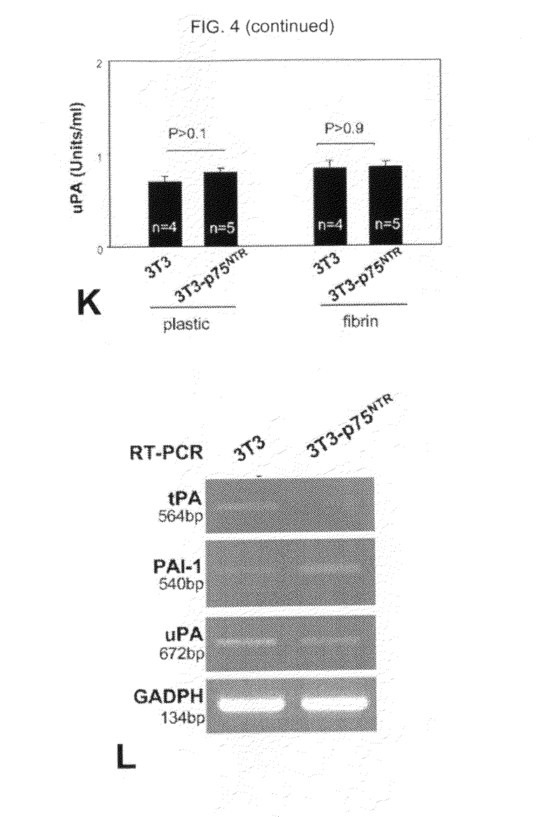

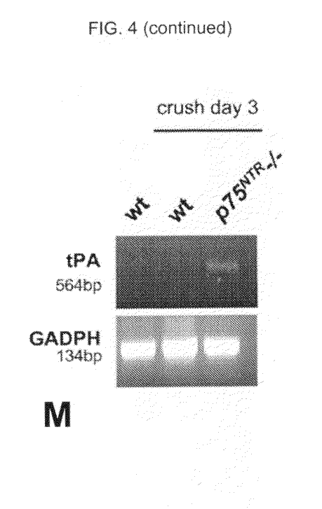

After finding a biological function for p75NTR in the regulation of tPA using SCs and sciatic nerves from p75NTR−/− mice, stable and transient transfections of p75NTR as well as siRNA against p75NTR were used to test the properties of p75NTR in heterologous systems. FIG. 4 depicts 3D fibrin gel degraded by NIH3T3 (a), but not by NIH3T3p75NTR cells (b). (c) Quantification of fibrin degradation. Experiments were performed seven times in duplicates. Phase-contrast microscopy shows lytic zones in NIH3T3 (d), but not in NIH3T3p75NTR cultures (e). Zymography shows degradation of casein by NIH3T3 cells (f), whereas NIH3T3p75NTR cells do not degrade casein (g). p75NTR blocking antibody (REX) in NIH3T3 (h) and NIH3T3p75NTR cells (i). Quantification of tPA (j) and uPA (k) activity in supernatants from NIH3T3 and NIH3T3p75NTR cultures. Experiments were performed five times in duplicates. (l) RT-PCR analysis for tPA, PAI-1, uPA, and GAPDH on cDNA derived from NIH3T3 and NIH3T3p75NTR cells. (m) RT-PCR analysis for tPA and GAPDH on cDNA derived from uninjured wt, and wt or p75NTR−/− mice three days after nerve injury. Bar graphs represent means±SEM (statistics by t test). Bar: 1.2 cm (a and b), 130 μm (d-g). These data suggest that neurotrophin/p75NTR signaling is not involved in the regulation of tPA in SCs and fi broblasts and that regulation of tPA by p75NTR is independent of neurotrophins.

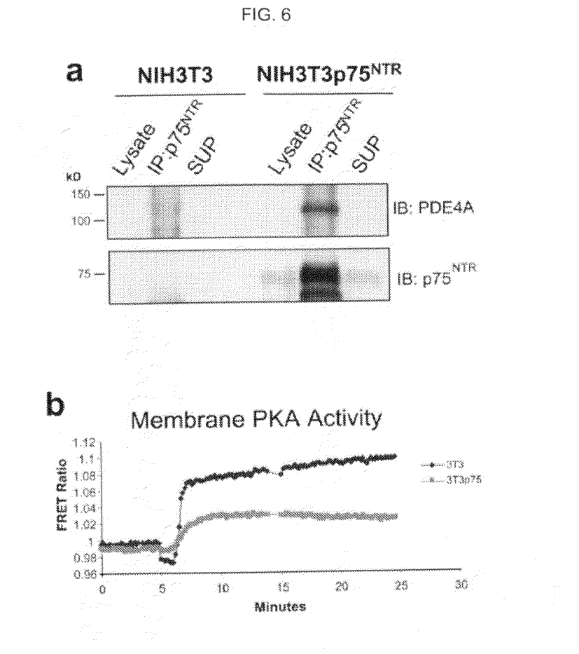

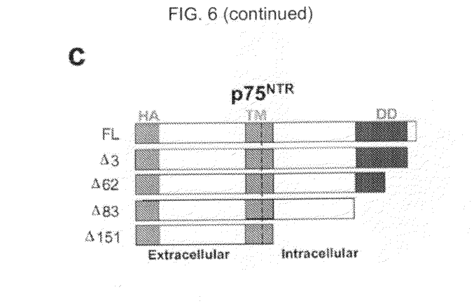

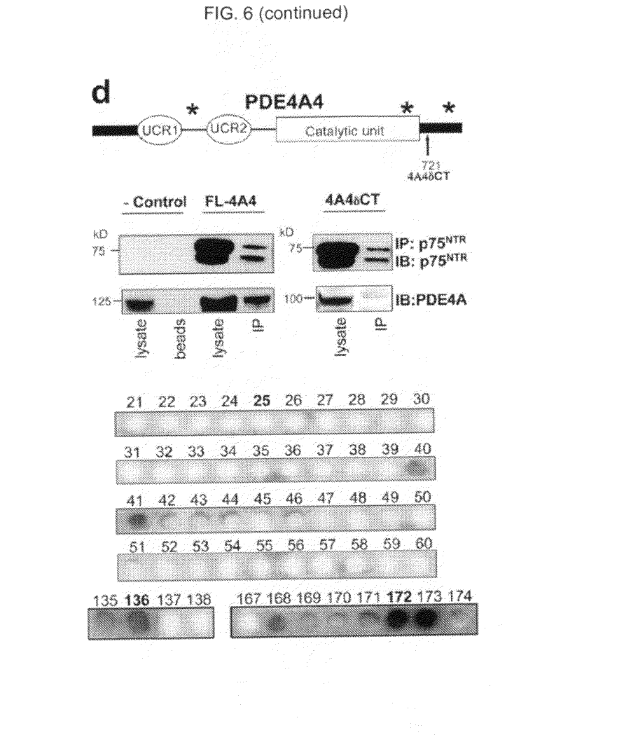

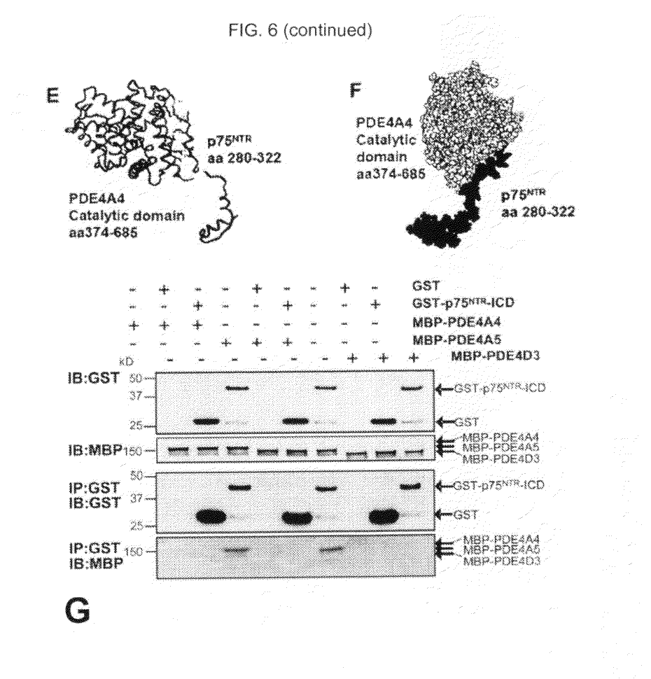

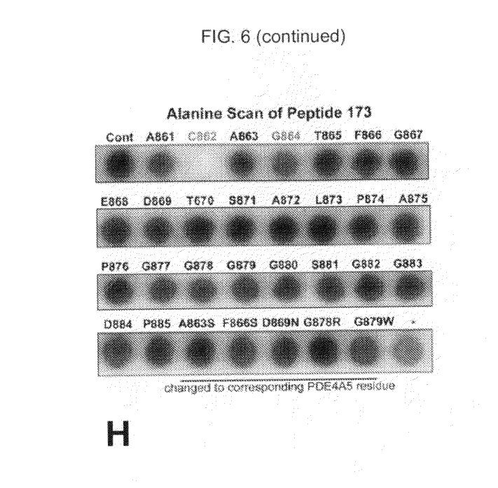

To examine whether p75NTR could inhibit fibrin degradation, NIH3T3 fibroblasts were stably transfected with p75NTR that exhibit high levels of p75NTR (105 receptors/cell) (Hsu and Chao, 1993). NIH3T3 cells on a 3D fibrin gel degrade fibrin (FIG. 4 a), whereas NIH3T3p75NTR cells do not (FIG. 4 b). Expression of p75NTR inhibits fibrin degradation by 12-fold (FIG. 4 c; P<0.001). NIH3T3 cells form lytic areas (FIG. 4 d), whereas NIH3T3p75NTR cells grow uniformly on fibrin (FIG. 4 e). NIH3T3 cells fully degrade the plasmin substrate casein (FIG. 4 f) but NIH3T3p75NTR cells do not degrade casein (FIG. 4 g), suggesting impaired proteolysis in NIH3T3p75NTR cells. Aprotinin, a general inhibitor of serine proteases, completely inhibits fibrin degradation by NIH3T3 cells (not depicted). In fibroblasts both tPA and uPA are involved in activation of plasminogen and fibrin degradation. tPA activity is significantly decreased in the NIH3T3p75NTR cells (FIG. 4 h). In contrast, expression of p75NTR has no effect on uPA activity (FIG. 6 i). tPA is a transcriptionally regulated immediate-early gene (Qian et al., 1993). Indeed, expression of p75NTR down-regulates tPA transcripts (FIG. 4 j). In addition, mRNA of PAI-1 is also upregulated in NIH3T3p75NTR cells (FIG. 4 j). Real-time quantitative PCR shows a 10.1-fold decrease in tPA mRNA, a fourfold increase in PAI-1 mRNA, and a twofold decrease in uPA mRNA in NIH3T3p75NTR cells. Upon expression of p75NTR, the decrease of uPA RNA does not affect uPA activity (FIG. 4 i). In contrast, the decrease of tPA RNA in NIH3T3p75NTR cells results in a corresponding decrease in tPA activity (FIG. 4 h; P<0.01).

After injury, sciatic nerves of p75NTR−/− mice show a fourfold increase of tPA RNA when compared with wt (FIG. 4 k). Moreover, p75NTR−/− mice show an increase in tPA RNA in primary cerebellar granule neurons (CGNs) (FIG. 10 c), and increased proteolytic activity in the cerebellum (FIG. 10, a and b). FIG. 10 depicts (a) In situ zymographies on cerebella from wt (n=6) and p75NTR−/− (n=5) mice reveal enhanced proteolytic activity in p75NTR−/− cerebella compared to wt. Quantification is shown in (b). (c). Quantitative real time PCR analysis of mRNA isolated from primary CGNs from wild-type and p75NTR−/− animals revealed a 4-fold increase in tPA levels in p75NTR−/− neurons.

Overall, these data suggest that expression of p75NTR inhibits the tPA/plasmin system both in vivo in the cerebellum and after sciatic nerve injury, as well as in vitro in primary neurons, SCs, as well as fibroblasts.

### Example 6

### p75NTR Regulates tPA and PAI-1 via a PDE4/cAMP/PKA Pathway

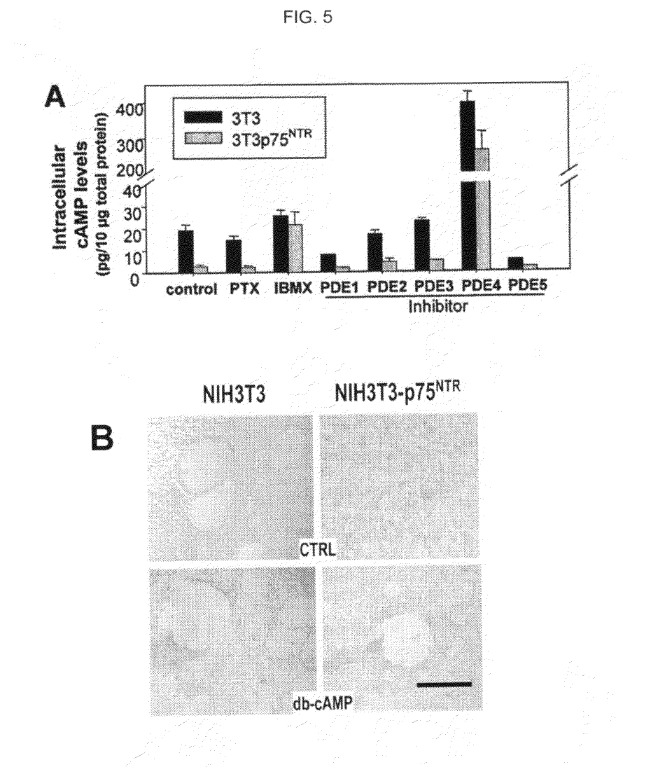

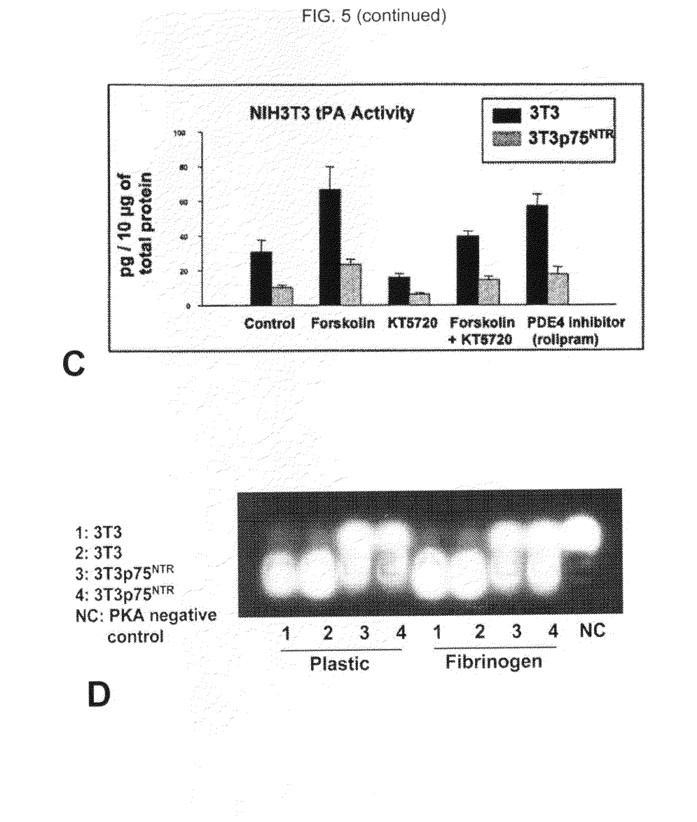

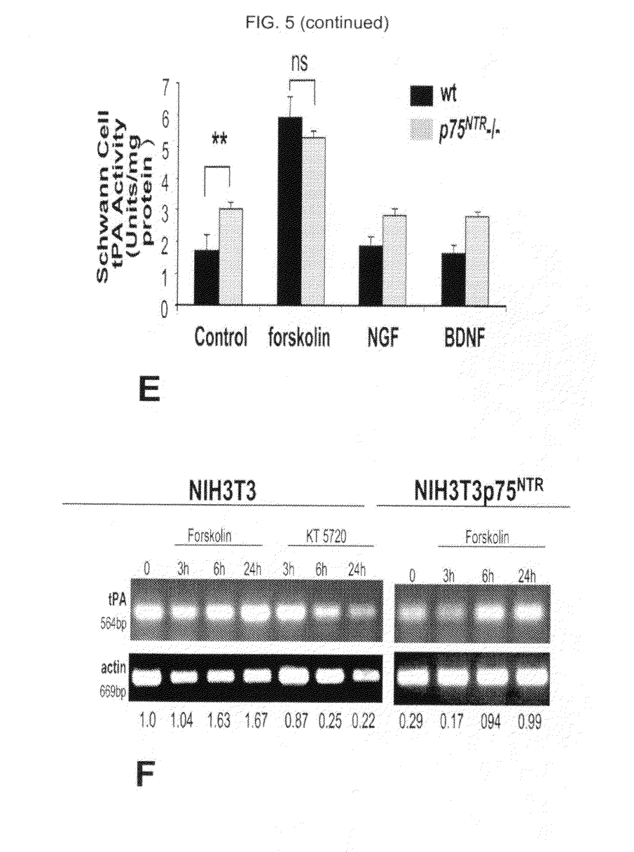

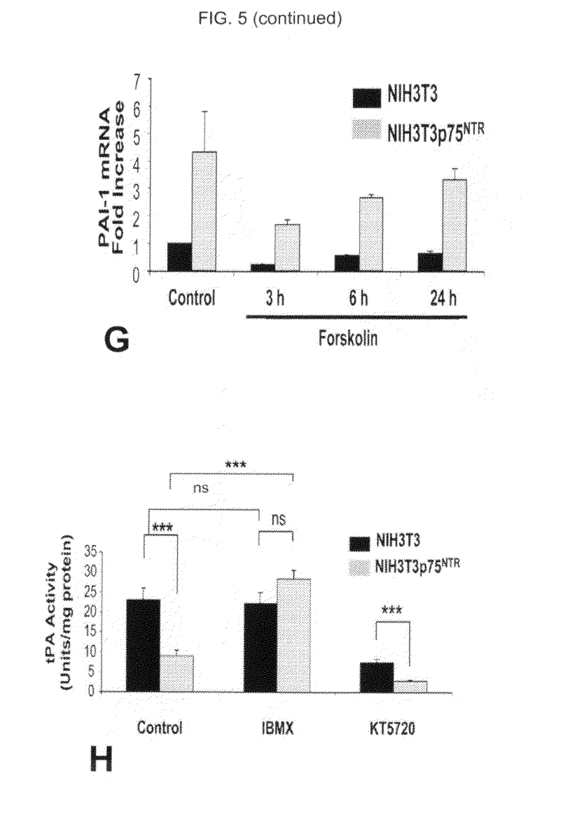

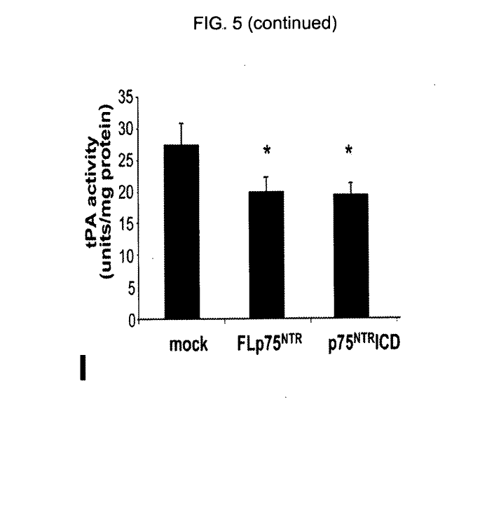

Transcriptional regulation of tPA depends on the cAMP/PKA pathway (Medcalf et al., 1990). FIG. 5 depicts (a) Intracellular cAMP levels in NIH3T3 and NIH3T3p75NTR fibroblasts shows a reduction of intracellular cAMP in NIH3T3 p75NTR cells, when compared to NIH3T3 cells (P<0.0001). Treatment with PTX, IBMX, specific inhibitors for PDE1, PDE2, PDE3, PDE4 (rolipram) and PDE5 shows that only IBMX (P<0.0001) and rolipram (P<0.0001) increase intracellular levels of cAMP in NIH3T3 p75NTR cells. (b) db-cAMP induces fibrinolysis in NIH3T3p75NTR cells. (c) Forskolin increases tPA activity in NIH3T3 fibroblasts, when compared to control NIH3T3 cells (P<0.02), and increases tPA activity of NIH3T3 p75NTR fibroblasts to the levels of NIH3T3 cells (P>0.4). Inhibition of PDE4 by rolipram shows an increase of tPA levels in NIH3T3p75NTR cells when compared to untreated NIH3T3p75NTR cells (P<0.001). Inhibition of PKA by KT5720 shows decrease of tPA activity in both NIH3T3 (P<0.005) and NIH3T3p75NTR fibroblasts (P<0.02). (d) IBMX increases tPA activity of NIH3T3p75NTR cells to the levels of NIH3T3 cells. Inhibition of PKA by KT5720 shows decrease of tPA activity in both NIH3T3 and NIH3T3p75NTR cells (P<0.0001). (e) PKA activity assay shows decrease of PKA in NIH3T3p75NTR cells. (f) Forskolin increases tPA mRNA in NIH3T3 and NIH3T3p75NTR cells. Inhibition of PKA by KT5720 decreases tPA transcript. (g) Quantification of PAI-1 mRNA changes by real time PCR shows a fourfold increase of PAI-1 mRNA in NIH3T3p75NTR cells compared with NIH3T3 cells. (h) Forskolin increases tPA activity in both wt (P<0.001) and p75NTR−/− (P<0.00001) SCs. NGF and BDNF do not affect activity of tPA (P>0.8 and P>0.3, respectively). (i) IBMX increases tPA activity of NIH3T3p75NTR cells to the levels of NIH3T3 cells. Inhibition of PKA by KT5720 shows decrease of tPA activity in both NIH3T3 and NIH3T3p75NTR cells (P<0.0001). (j) Transient overexpression of FL p75NTR or p75 ICD leads to decreased levels of tPA in NIH3T3 cells. Experiments were performed at least 5 times in duplicates. *, P<0.0001; **, P<0.05; ***, P<0.01. NS: non-significant. Bar graphs represent means±SEM (statistics by ANOVA).

Indeed, elevation of cAMP, using dibutyryl-cAMP (db-cAMP), overcomes the inhibitory effect of p75NTR (FIG. 5 a). Moreover, cAMP elevation, elicited using the general PDE inhibitor IBMX, elevates tPA activity in NIH3T3p75NTR to the levels seen in NIH3T3 cells (FIG. 5 b). IBMX does not affect basal levels of tPA in NIH3T3 cells (FIG. 5 b). These data suggest that PDE activity is required for the p75NTR induced tPA decrease.

PKA activity is decreased in NIH3T3p75NTR cells (FIG. 5 c, lanes 3 and 4) compared with NIH3T3 cells (FIG. 5 c, lanes 1 and 2), suggesting that p75NTR expression reduces PKA activity. KT5720, a specific PKA inhibitor, decreases tPA activity in NIH3T3 cells (FIG. 5 b). Because the cAMP/PKA pathway enhances tPA transcription and suppresses PAI-1 secretion (Santell and Levin, 1988), the cAMP/PKA pathway was tested for influences of p75NTR regulation of tPA and PAI-1. Forskolin-induced cAMP elevation increases, whereas KT5720-induced PKA inhibition decreases tPA RNA in NIH3T3 cells (FIG. 5 d). Forskolin treatment of NIH3T3p75NTR cells also increases both tPA RNA (FIG. 5 d) and activity (not depicted), whereas forskolin decreases PAI-1 RNA in both NIH3T3 and NIH3T3p75NTR cells (FIG. 5 e).

Similar to NIH3T3 cells, elevation of cAMP increases the activity of tPA in both wt and p75NTR−/− SCs (FIG. 5 f). Brainderived neurotrophic factor (BDNF)/TrkB signaling has been shown to regulate tPA in primary cortical neurons (Fiumelli et al., 1999). In contrast to cortical neurons, SCs are known to express minute levels of TrkB but high levels of p75NTR (Cosgaya et al., 2002). As provided herein, treatment of SCs with either BDNF or nerve growth factor (NGF) has no effect on tPA (FIG. 5 f). Similar results are obtained after treatment of SCs with pro-NGF, the high-affinity ligand of p75NTR (Lee et al., 2001) (unpublished data). In addition, in NIH3T3 and NIH3T3p75NTR cells, which do not express Trk receptors, the p75NTR-mediated suppression of tPA activity occurs independent of neurotrophins or serum (unpublished data). In accordance, in NIH3T3 cells transient expression of the intracellular domain (ICD) of p75NTR decreases tPA similar to the full-length (FL) p75NTR (FIG. 5 g).

### Example 7

### p75NTR Decreases cAMP Via PDE4

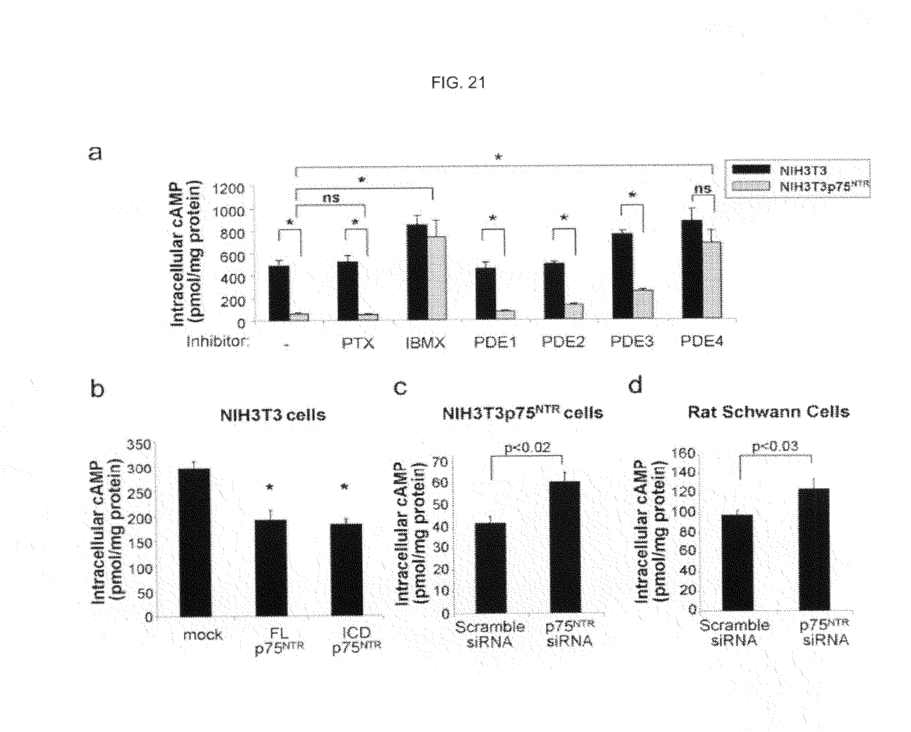

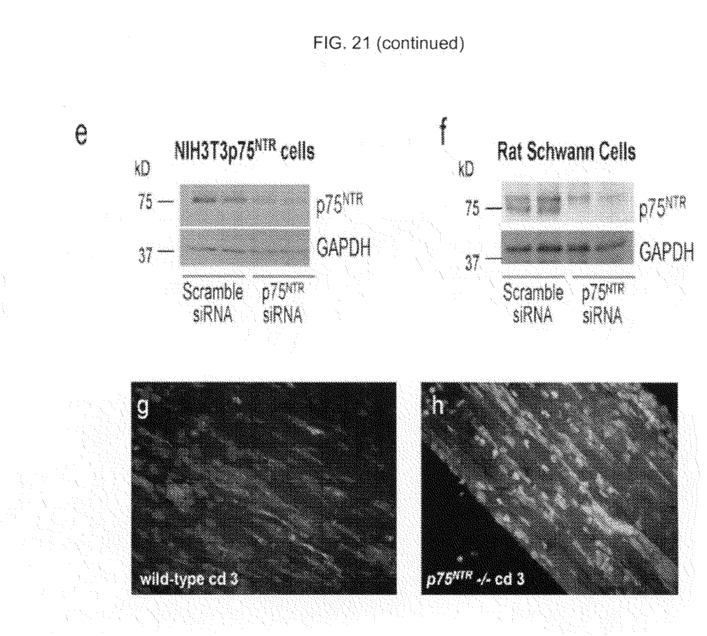

Because the effects of p75NTR were overcome by elevating cAMP, p75NTR was examined to determine whether it reduced cAMP levels. FIG. 21 depicts (a) cAMP levels in NIH3T3 and NIH3T3p75NTR cells show a reduction of cAMP in NIH3T3p75NTR cells, as compared with NIH3T3 cells. Treatment with PTX, IBMX, specific inhibitors for PDE1, PDE2, PDE3, and PDE4 (rolipram) shows that only IBMX (IC50 for PDE4 2-50 μM) and rolipram (IC50 for PDE4 0.8 μM) (P<0.0001) increase levels of cAMP in NIH3T3p75NTR cells to the levels of NIH3T3 cells. (b) Transient overexpression of FL p75NTR or p75 ICD leads to decreased levels of cAMP in NIH3T3 cells. (c) siRNA mediated knockdown of p75NTR levels in NIH3T3p75NTR cells leads to increased levels of cAMP. (d) siRNA mediated knockdown of p75NTR in primary rat Schwann cells leads to increased levels of cAMP. p75NTR levels after siRNA knock down in duplicate samples of NIH3T3p75NTR cells (e) and SCs (f). Immunostaining to detect cAMP in injured sciatic nerve reveals increased cAMP immunoreactivity in the sciatic nerve of p75NTR−/− mice (h) when compared with wt controls (g). Experiments were performed four times in duplicate. Bar graphs represent means±SEM (statistics by ANOVA or t test).

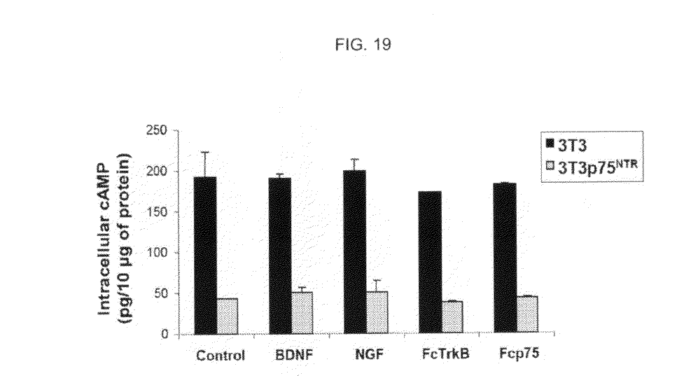

Indeed, cAMP is decreased 7.8-fold in NIH3T3p75NTR cells (FIG. 21 a; P<0.0001). Transient expression of p75NTR in NIH3T3 cells decreases levels of cAMP (FIG. 21 b; P<0.0005). Furthermore, siRNA knockdown against p75NTR leads to increased cAMP levels in both NIH3T3p75NTR cells (FIG. 21, c and e; P<0.02) and primary SCs (FIG. 21, d and f; P<0.03). NIH3T3 cells transiently transfected with p75NTR express fivefold less p75NTR than the stably transfected NIH3T3p75NTR cells (unpublished data). Differences in expression between stably and transiently transfected cells may account for the differences in the fold-decrease of cAMP and tPA between these two systems. Moreover, immunostaining with an antibody against cAMP shows increased cAMP in injured sciatic nerves from p75NTR−/− mice (FIG. 21, g and h). In neurons BDNF elevates cAMP exclusively via TrkB (Gao et al., 2003). In NIH3T3p75NTR cells, which do not express TrkB, stimulation with NGF or BDNF does not affect the p75NTR-mediated suppression of cAMP (FIG. 19). FIG. 19 depicts expression of p75NTR is sufficient for the reduction of intracellular cAMP (control). Addition of neurotrophins, such BDNF or NGF or inhibition of neurotrophins in the cell culture medium either by Fc-TrkB or Fc-p75NTR does not affect the levels of intracellular cAMP in either NIH3T3 or NIH3T3p75NTR cells. Experiments were performed five times in duplicates.

Similarly, inhibition of neurotrophins by Fc-p75NTR or BDNF by Fc-TrkB does not alter cAMP levels in NIH3T3p75NTR cells (FIG. 19). In accordance, transient expression of the ICD of p75NTR decreases cAMP similar to the FL p75NTR in NIH3T3 cells (FIG. 21 b). Overall, these data suggest a neurotrophin-independent PDE4/cAMP pathway downstream of p75NTR, which consequently leads to decreases in extracellular proteolysis.

Down-regulation of cAMP can be mediated either by inhibition of cAMP synthesis via the action of Gi, a G protein that inhibits adenylyl cyclase or via the action of PDEs. Treatment of cells with pertussis toxin (PTX) that blocks interactions between the Gi and G protein coupled receptors, does not rescue the p75NTR-mediated down-regulation of cAMP (FIG. 21 a; P>0.5). In contrast, the PDE inhibitor IBMX resulted in significant increase of cAMP in the NIH3T3p75NTR cells when compared with control NIH3T3p75NTR cells (FIG. 21 a; P<0.000001). Use of specific chemical inhibitors for PDE isoforms shows that only rolipram, a specific inhibitor of PDE4, significantly increases cAMP levels in NIH3T3p75NTR cells (FIG. 21 a; P<0.000001) to the levels of NIH3T3 cells (FIG. 21 a; P=0.051), suggesting that the p75NTR-induced cAMP decrease is mediated via PDE4.

### Example 8

### p75NTR Targets cAMP Degradation to the Membrane Via Direct Recruitment of PDE4A5

Recruitment of PDE4 to subcellular structures such as the plasma membrane concentrates the activity of PDEs and reduces PKA activity by enhancing degradation of cAMP (Brunton, 2003; Houslay and Adams, 2003). FIG. 6 depicts (a) Endogenous PDE4A5 co-IPs with p75NTR in NIH3T3p75NTR cells. Lysates were immunoprecipitated with anti-p75NTR and probed with anti-PDE4A or anti-p75NTR. Due to the low endogenous levels of PDE4A, higher exposure was necessary to detect PDE4A5 in the lysates (see FIG. S3 c). (b) FRET emission ratio change of NIH3T3 and NIH3T3p75NTR cells for the pm-AKAR2.2 in response to forskolin. FRET change represents membrane activation of PKA (c) Mapping of the p75NTR sites required for interaction with PDE4A5. Schematic diagram of HA-tagged p75NTR intracellular deletions. TM, transmembrane domain; DD, death domain. Lysates were immunoprecipitated with an anti-HA antibody and probed with anti-PDE4A or anti-p75NTR. (d) Mapping of the PDE4A4 sites required for interaction with p75NTR. Schematic diagram of the C-terminal deletion of PDE4A4. Arrow indicates the deletion site. Lysates were immunoprecipitated with anti-p75NTR and probed with anti-PDE4A or anti-p75NTR. Computer simulated docking ribbon (e) and CPK (f) models of the catalytic domain of PDE4A4 with the p75NTR ICD. The residues of PDE4A4 shown to interact with p75NTR ICD in silico are found to be within the same interacting sequences identified in vitro using peptide arrays and coimmunoprecipitation. (g) Co-IP of purified, recombinant proteins reveals that both PDE4A4 and PDE4A5 interact with the ICD of p75NTR, but PDE4D3 does not. (h) PDE4A4 peptide library screened with recombinant GST-p75NTR ICD revealed three distinct domains of PDE4A4 (asterisks in d) that interact with the ICD of p75NTR: the LR1 domain (peptides 40 and 41), the catalytic domain (peptides 135 and 136) and the unique C terminus (peptides 172 and 173). (i) Alanine scanning mutagenesis shows that substitution of C862 abolishes the interaction of p75NTR with the 173 peptide that is unique to PDE4A.

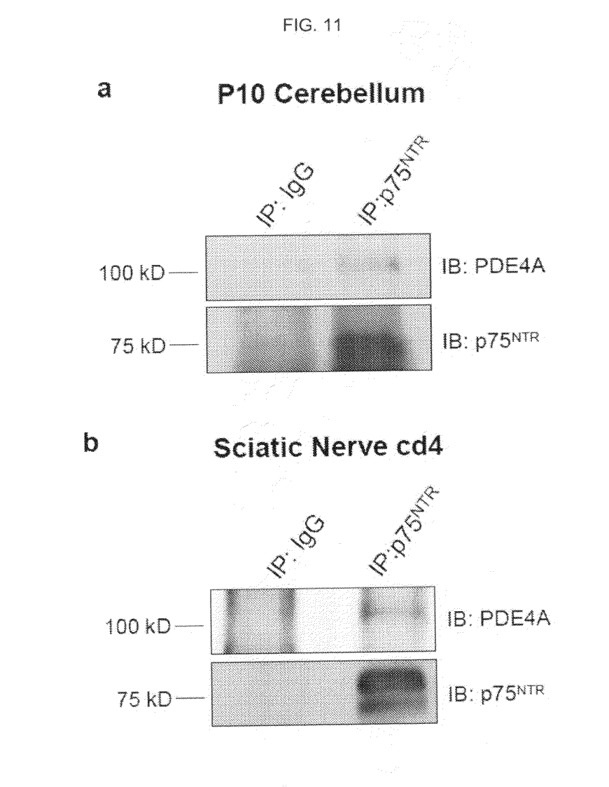

p75NTR was examined to determine whether it regulates cAMP via recruitment of PDE4. In NIH3T3p75NTR cells, p75NTR coimmunoprecipitates (co-IPs) with endogenous PDE4A (FIG. 6 a). No association is observed with the other three PDE4 sub-families, namely PDE4B, PDE4C, or PDE4D (unpublished data), suggesting that the effect was PDE4A specific. Based on the molecular weight of PDE4A at 109 kD, it was determined that p75NTR co-IPs with the PDE4A5 isoform. FIG. 11 depicts Communoprecipitation (IP) experiments from wild-type CGNs reveal that endogenous levels of PDE4A5 and p75NTR are able to form a complex in wild-type CGNs (a). IP with rabbit IgG is used as negative control. Co-IP experiments from crushed wild-type sciatic nerves reveal that endogenous levels of PDE4A5 and p75NTR are able to form a complex in the injured sciatic nerve as well (b). Western blot demonstrating similar levels of PDE4A5 expression in NIH3T3 and NIH3T3p75NTR cells (c).

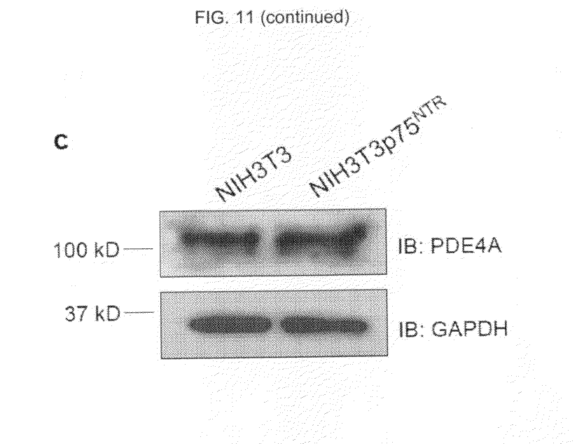

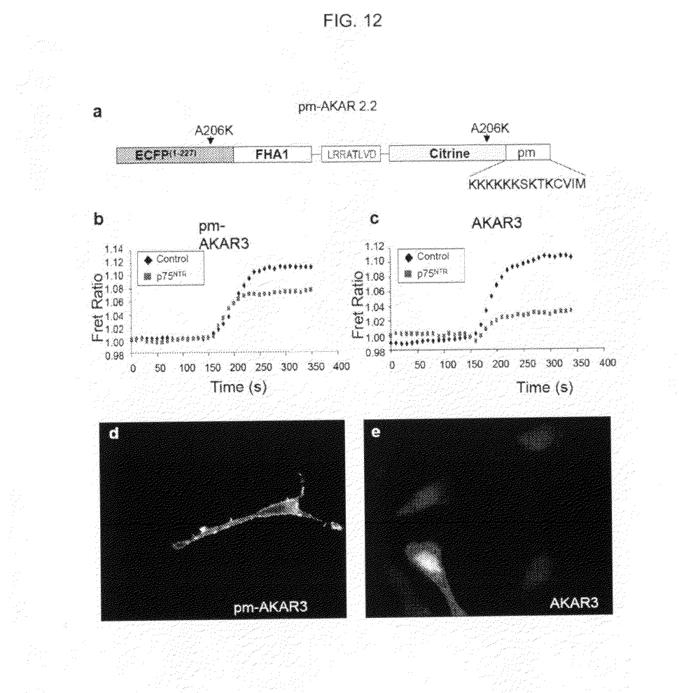

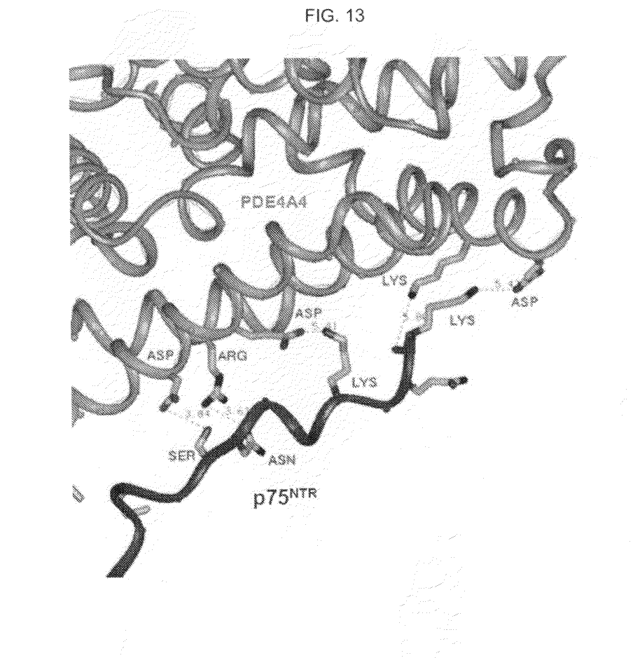

Endogenous co-IP in CGNs (FIG. 11) and in injured sciatic nerve (FIG. 11 b) shows that p75NTR and PDE4A5 interact at endogenous expression levels. Analysis of lysates shows that the levels of PDE4A are similar in NIH3T3 and NIH3T3p75NTR cells (FIG. 11 c). These results show that p75NTR forms a complex with PDE4A5. A functional consequence of the p75NTR-PDE4A5 interaction would be recruitment of PDE4A5 to the membrane resulting in decreased membrane-associated cAMP/PKA signaling. FIG. 12 depicts (a) Generation of plasma membrane targeted PKA fluorescent indicator (pm-AKAR 2.2) Domain structures of pm-AKAR2.2. The C-terminal sequence from K-Ras KKKKKKSKTKCVIM, containing a six lysine repeat and a CAAX box, was added to target the construct to the plasma membrane. ECFP, enhanced cyan fluorescent protein; FHA1, forkhead associated domain 1; LRRATLVD, PKA substrate sequence; Citrine, an improved version of yellow fluorescent protein; pm, plasma membrane targeting sequence. pm-AKAR2.2 is a novel membrane-targeted fluorescent reporter of PKA activity that could be a useful tool in studying spatial and temporal regulation of cAMP/PKA signaling in living cells. FRET emission ratio change of control NIH3T3 cells and NIH3T3 cells transiently transfected with p75NTR and co-transfected with the pm-AKAR3 (b) or AKAR3 (c) in response to forskolin, which activates adenylyl cyclase at the plasma membrane. Images show the localization for pm-AKAR3 (d) and AKAR3 (e). pm-AKAR3 localizes at the membrane (d). Experiments for (b) and (c) were performed three times in triplicates.

To investigate whether p75NTR reduces membrane-associated PKA activity, the genetically encoded A-kinase activity reporter was modified, AKAR2 (Zhang et al., 2005) and generated pm-AKAR2.2, a membrane-targeted fluorescent reporter of PKA activity that generates a change in fluorescence resonance energy transfer (FRET) when it is phosphorylated by PKA in living cells (FIG. 12 a). As expected, NIH3T3 cells show a dramatic emission ratio change for the pm-AKAR2.2 in response to forskolin (FIG. 6 b). In contrast, NIH3T3p75NTR cells show an attenuated response, revealing reduced PKA activity at the plasma membrane (FIG. 6 b). Transient transfection of p75NTR confirmed the results observed in the stable NIH3T3p75NTR cells using the latest generation of plasma membrane-specific PKA biosensor AKAR3 (Allen and Zhang, 2006) (FIG. 12 b). As expected, increased cAMP degradation at the plasma membrane results in decreased intracellular cAMP (FIG. 12 c; FIG. 5, a and b). Overall, the results showing reduced membrane-associated PKA activity upon expression of p75NTR suggest that p75NTR targets cAMP degradation to the membrane via its interaction with PDE4A5. To verify the specificity of p75NTR-PDE4A5 association, a series of mapping studies were conducted using deletion mutants. PDE4A5 interacts with FL p75NTR, as well as deletions Δ3, Δ62, Δ83, but not a deletion missing the distal 151 amino acids, Δ151 (FIG. 6 c), suggesting that the interaction between p75NTR and PDE4A5 occurs in the juxtamembrane region of p75NTR, requiring sequences between residues 275 and 343. To explain the specificity of the interaction of p75NTR with a single PDE4 isoform, p75NTR is provided to interact with a unique region of PDE4A5 that is not present in other PDE4s. Although the PDE4 isoforms are highly homologous, PDE4A5 contains a unique C-terminal region with a yet unknown biological function (Houslay and Adams, 2003). Co-IP experiments in HEK293 cells using the PDE4A4δCT mutant that is missing the C-terminal region (aa 721-886) abolishes the interaction with p75NTR (FIG. 6 d). To examine whether p75NTR could interact with PDE4A5 in a direct manner, in vitro pull-down assays were performed using recombinant proteins. A GST fusion protein of p75NTR encoding the entire ICD interacts with both recombinant PDE4A5 and its human homologue PDE4A4 (FIG. 6 e). In contrast, p75NTR ICD does not interact with recombinant PDE4D3 (FIG. 6 e). These results are in accordance with both the endogenous co-IPs in cells (FIG. 6, a and c; FIG. 11) and the PDE4A4 mutagenesis data (FIG. 6 d) because similar to PDE4A4δCT, PDE4D3 does not contain the unique C-terminal domain of PDE4A4/5. peptide array technology was used to define sites of direct interaction in other PDE4s (Bolger et al., 2006). Screening a peptide array library of overlapping 25-mer peptides that scanned the sequence of PDE4A4 with GST-ICD p75NTR identified interactions with the LR1 domain, whose sequence is unique to the PDE4A subfamily (peptides 40 and 41, aa 191-220), and also to a sequence within the catalytic domain (peptides 135 and 136, aa 671-700). However, the strongest interaction was observed with sequences within the C-terminal region of PDE4A4 (peptides 172 and 173, aa 856-885). Alanine scanning mutagenesis shows that substitution of C862 abolishes the interaction of p75NTR with the 173 peptide that is unique to PDE4A (FIG. 6 g). The p75NTR-interacting sequences within the LR1 and C-terminal domains are highly conserved between the human PDE4A4 and the rodent PDE4A5. Indeed, peptide array screening for PDE4A5 reveals direct interaction with p75NTR similar to that seen for PDE4A4 (unpublished data). Overall, these results suggest that the interaction of p75NTR with PDE4A4/5 is direct and that sequences within the juxtamembrane region of p75NTR and the unique C-terminal region of PDE4A4/5 are primarily required for the interaction (FIG. 6, a, c-g; FIG. 11).

### Example 9

### p75NTR Regulates Plasminogen Activation and Fibrin Deposition as a Model of LPS-Induced Pulmonary Fibrosis

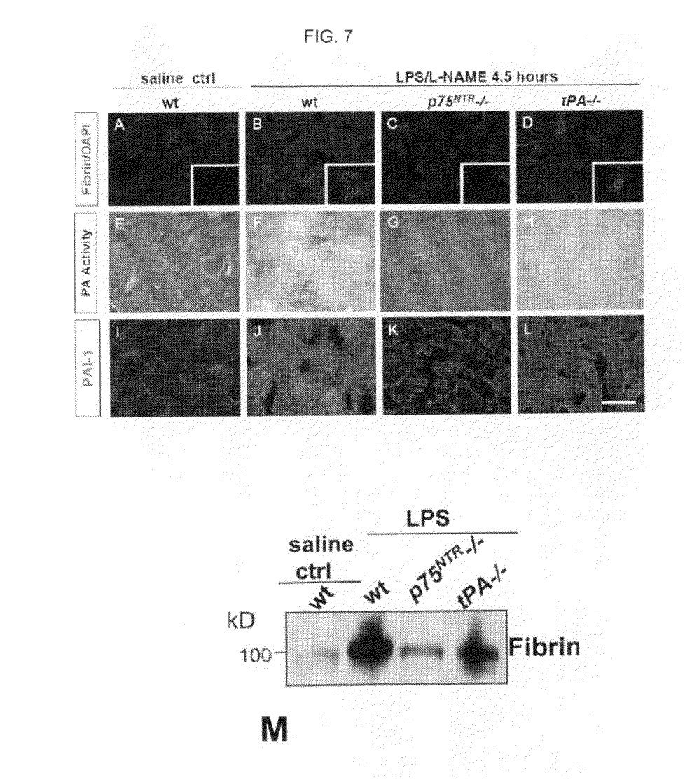

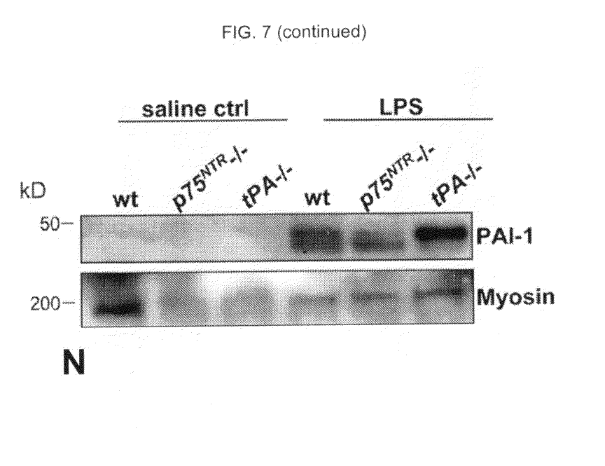

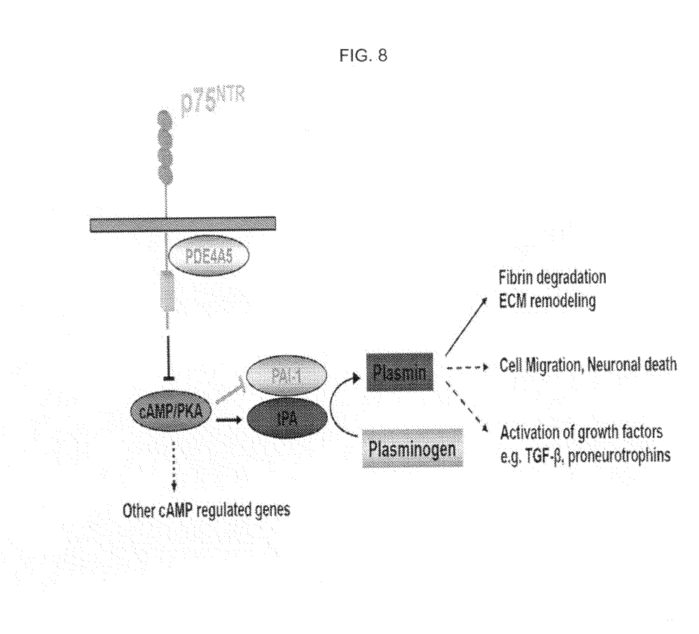

Because expression of p75NTR inhibits fibrinolysis in fibroblasts, the role of p75NTR is provided to be a modulator of fibrinolysis extending to tissues outside of the nervous system that express p75NTR after injury or disease. Because p75NTR is expressed in the lung (Ricci et al., 2004), the levels of fibrin in the lung of wt and p75NTR−/− mice were compared in a model of lipopolysaccharide (LPS)-induced lung fibrosis (Chen et al., 2004). FIG. 7 depicts LPS induces fibrin deposition (red) in the wt lung (b), when compared with the saline-injected lung (a). Lungs derived from p75NTR−/− mice show less fibrin deposition (c). In situ zymography after 3 h of incubation shows clearance of casein in the lung of saline-injected wt (d), when compared with LPS injected wt lung (e). Lung from LPS-treated p75NTR−/− mouse shows enhanced proteolytic activity (f), when compared with the wt mouse (e). Immunoreactivity for PAI-1 is increased in wt lung derived from LPS-treated mouse (h), when compared with saline-treated control (g). Lung from LPS-treated p75NTR−/− mouse shows decreased PAI-1 (i), when compared with the wt LPS-treated mouse (h). (j) Western blot of fibrin precipitation from the lung shows an up-regulation of fibrin in the LPS-treated wt lung, when compared with the p75NTR−/− lung. (k) Western blot for PAI-1 in the lung shows a decrease of PAI-1 in the p75NTR−/− lung, when compared with the wt lung. Images are representative of n=10 wt and n=9 p75NTR−/− mice. Western blots have been performed for n=4 wt and n=4 p75NTR−/− mice. Bar: 150 μm (a-c), 75 μm (a-c, inset), 200 μm (d-f), 150 μm (g-i).

LPS-treated wt mice showed widespread extravascular fibrin deposition (FIG. 7 b) and decreased proteolytic activity after LPS treatment (FIG. 7 e), when compared with saline-treated wt mice (FIG. 7, a and d). In contrast, p75NTR−/− mice show a 2.58-fold decrease of fibrin immunoreactivity (FIG. 7, c and j) and increased proteolytic activity (FIG. 7 f). Decreased proteolytic activity in the lung after injury depends on the up-regulation of PAI-1 (Idell, 2003). Loss of PAI-1 protects from pulmonary fibrosis in LPS-induced airway disease, hyperoxia, and bleomycin-induced fibrosis (Savoy et al., 2003). Because p75NTR increases PAI-1 (FIG. 5 j and FIG. 7 e), p75NTR was shown to regulate expression of PAI-1 in vivo. PAI-1 is up-regulated in LPS-treated wt mice (FIG. 7 h) when compared with saline-treated wt mice (FIG. 9 g). FIG. 9 depicts Increased fibrin deposition in the crushed sciatic nerve of p75NTR−−tPA−/− mice (c), when compared with crushed p75NTR−/− sciatic nerve (b). Wt (a) and tPA−/− (d) nerves are used for control. In situ zymography shows lack of proteolytic activity in the crushed p75NTR−/−tPA−/− sciatic nerves (n=5) (g), when compared with crushed p75NTR−/− sciatic nerves (n=20) (f). Crushed wt (e) and tPA−/− (h) nerves are used for control. Fibrin immunostainings and zymographies were performed on n=5 p75NTR−/−tPA−/−, n=20 p75NTR−/−, n=20 wt, n=5 tPA−/− mice. Representative images are shown. (i) Quantification of proteolytic activity 4 d after crush injury shows statistically significant increase for proteolytic activity in p75NTR−/− mice. Quantification results are based on n=5 p75NTR−/−, n=5 p75NTR−/−tPA−/−, n=5 tPA−/− and n=4 wt mice. Bar graph represents means±SEM (*, P<0.05; by ANOVA). Bar: 50 μm (a-d), 300 μm (e-h).

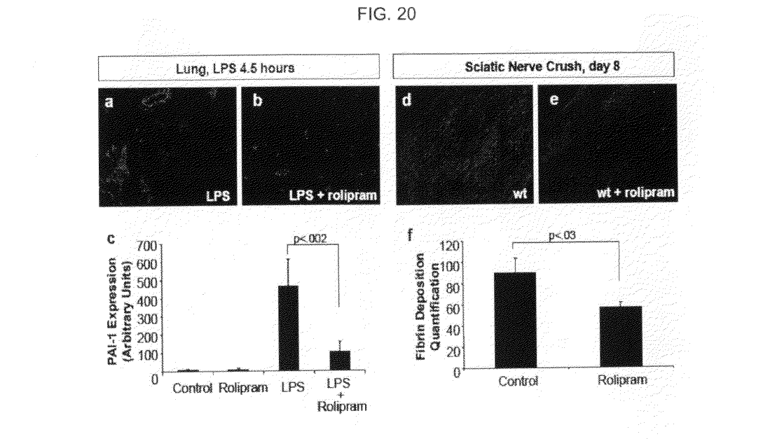

In contrast, LPS-treated p75NTR−/− mice show similar immunoreactivity for PAI-1 (FIG. 9 i) as saline-treated wt mice (FIG. 7 g), suggesting that p75NTR induces up-regulation of PAI-1 after injury in the lung. Western blots show a decrease in PAI-1 in the lungs of p75NTR−/− mice (FIG. 7 k). FIG. 20 depicts Fibrin deposition (red) is decreased in the lung in rolipram treated mice after induction of LPS-induced acute lung injury (b), when compared to mice treated with LPS alone (a). Quantification shows a 34% decrease in fibrin rolipram vs. control treated wt lungs after LPS-induced lung fibrosis (not shown). Quantitative PCR of PAI-1 transcripts show a reduction of LPS-induced PAI-1 upregulation after rolipram treatment, but no effect of rolipram treatment alone (c). S Rolipram treatment led to decreased levels of fibrin deposition in wt nerves 8 days after sciatic nerve crush injury (e), when compared to untreated wt nerves (d). Quantification revealed statistically significant reduction of fibrin deposition after rolipram treatment (f). Quantification of the lung samples is based on n=7 LPS treated mice, n=4 LPS+rolipram treated mice, n=5 rolipram treated mice and n=7 control untreated mice. Quantification of the sciatic nerve samples is based on n=9 wt and n=9 wt+rolipram treated mice.

Similar to the p75NTR−/− mice, rolipram reduces fibrin deposition in the lung (FIG. 20, a and b) and sciatic nerve (FIG. 20, d-f), and decreases PAI-1 in the lung (FIG. 20 c), suggesting the involvement of PDE4 in p75NTR-mediated inhibition of fibrinolysis in vivo. Collectively, the data show that p75NTR increases fibrin deposition via a PDE4-mediated inhibition of plasminogen activation in both LPS-induced lung fibrosis and sciatic nerve crush injury. These data suggest a role for p75NTR/PDE4 signaling as a general regulator of plasminogen activation and fibrinolysis at sites of injury.

### Example 10

### p75NTR Downregulates cAMP by Targeting its Degradation to the Plasma Membrane

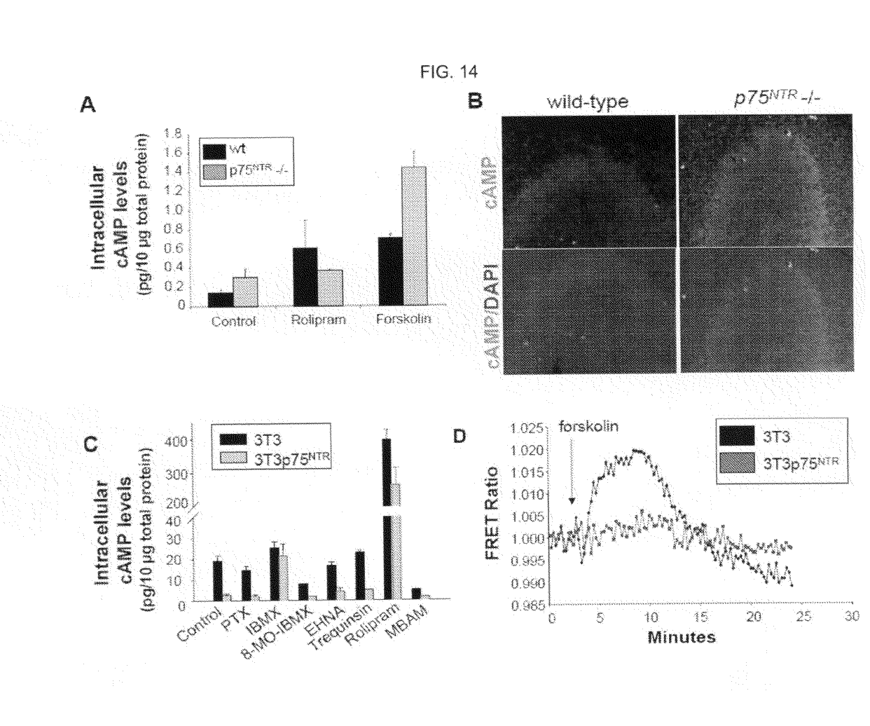

CGNs isolated from p75NTR−/− animals exhibit a two-fold increase in intracellular cAMP compared to wild-type controls both basally or after forskolin treatment (p<0.025) (see e.g., FIG. 14A). FIG. 14 depicts (A) CGNs isolated from p75NTR−/− animals exhibit a two-fold increase in intracellular cAMP compared to wild-type controls both basally or after forskolin treatment (p<0.025). Treatment of cells with rolipram, an inhibitor of PDE4s, leads to a four-fold increase in intracellular cAMP in wild-type cells (p<0.015), but no significant increase in p75NTR−/− cells (p=0.669). (B) Immunohistochemical staining of cerebella isolated from P10 mice revealed increased immunoreactivity of cAMP in the granular and molecular layers of p75NTR−/− animals when compared to wild-type controls. (C) Overexpression of p75NTR decreases intracellular cAMP in NIH3T3p75NTR cells, when compared to NIH3T3 cells (P<0.0001). Treatment with inhibitors of G proteins (pertussis toxin, PTX 100 ng/ml), a pan-phosphodiesterase inhibitor (IBMX, 500 μM), specific inhibitors for PDE1 (8-Methoxymethyl-IBMX, 18.7 μM), PDE2 (EHNA, 80 μM), PDE3 (trequinsin, 100 nM), PDE4 (rolipram, 10 μM) and PDE5 (4-{[3′,4′-(Methylenedioxy)benzyl]amino}-6-methoxyquinazoline, 23 μM) shows that only IBMX (P<0.0001) and rolipram (P<0.0001) increase intracellular levels of cAMP in NIH3T3p75NTR cells. (D) NIH3T3 and NIH3T3p75NTR cells were transfected with the plasma membrane targeted AKAR 2.2 PKA reporter construct and processed for live-cell imaging using fluorescent confocal microscopy. In response to forskolin stimulation, NIH3T3 cells exhibited an increase in cAMP-dependent kinase activity at the membrane, whereas, cells overexpressing p75NTR did not.

Treatment of cells with rolipram, an inhibitor of PDE4s, leads to a four-fold increase in intracellular cAMP in wild-type cells (p<0.015), but no significant increase in p75NTR−/− cells (p=0.669). Immunohistochemical staining of cerebella isolated from P10 mice revealed increased immunoreactivity of cAMP in the granular and molecular layers of p75NTR−/− animals when compared to wild-type controls (see e.g., FIG. 14B). Overexpression of p75NTR decreases intracellular cAMP in NIH3T3p75NTR cells, when compared to NIH3T3 cells (P<0.0001) (see e.g., FIG. 14C) Treatment with inhibitors of G proteins (pertussis toxin, PTX 100 ng/ml), a pan-phosphodiesterase inhibitor (IBMX, 500 μM), specific inhibitors for PDE1 (8-Methoxymethyl-IBMX, 18.7 μM), PDE2 (EHNA, 80 μM), PDE3 (trequinsin, 100 nM), PDE4 (rolipram, 10 μM) and PDE5 (4-{[3′,4′-(Methylenedioxy)benzyl]amino}-6-methoxyquinazoline, 23 μM) shows that only IBMX (P<0.0001) and rolipram (P<0.0001) increase intracellular levels of cAMP in NIH3T3p75NTR cells. NIH3T3 and NIH3T3p75NTR cells were transfected with the plasma membrane targeted AKAR 2.2 PKA reporter construct and processed for live-cell imaging using fluorescent confocal microscopy (see e.g., FIG. 14D). In response to forskolin stimulation, NIH3T3 cells exhibited an increase in cAMP-dependent kinase activity at the membrane, whereas, cells overexpressing p75NTR did not.

### Example 11

### p75NTR Co-Immunoprecipitates with PDE4A5 and the p75NTR Juxtamembrane Sequence (Arg275-Leu342) Associates with PDE4A5

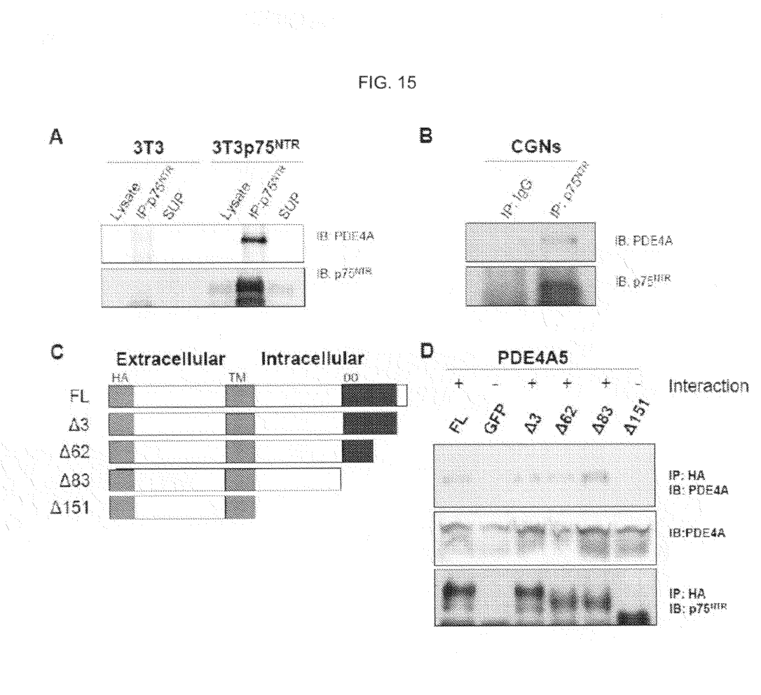

PDE4A5 co-immunoprecipitates with p75NTR in NIH3T3p75NTR fibroblasts (see e.g., FIG. 15A). FIG. 15 depicts p75NTR co-immunoprecipitates with PDE4A5 and the p75NTR juxtamembrane sequence (Arg275-Leu342) associates with PDE4A5 (A) PDE4A5 co-immunoprecipitates with p75NTR in NIH3T3p75NTR fibroblasts. Cell lysates were immunoprecipitated (IP) with an anti-p75NTR antibody (9992) and probed with an anti-PDE4A antibody to detect co-precipitated PDE4A. Cell lysates were probed with an anti-PDE4A or an anti-p75NTR antibody (9651) to detect the expression levels of PDE4A and p75 receptors, respectively. (B) Endogenous co-immunoprecipitation of p75NTR with PDE4A5 in primary CGNs was performed as in (A). (C) Mapping of the p75NTR sites required for interaction with PDE4A5. Schematic diagram of HA-tagged p75NTR intracellular deletions. TM represents the transmembrane domain. DD represents the cytoplasmic death domain. PDE4A5 was co-transfected with HA-tagged p75NTR deletion constructs into 293 cells. Cell lysates were immunoprecipitated (IP) with an anti-HA antibody and probed with an anti-PDE4A antibody to detect co-precipitated PDE4A. Cell lysates were probed with an anti-PDE4A or an anti-p75NTR antibody (9651) to detect the expression levels of PDE4A and p75 receptors, respectively. IB, Immunoblot.

Cell lysates were immunoprecipitated (IP) with an anti-p75NTR antibody (9992) and probed with an anti-PDE4A antibody to detect co-precipitated PDE4A. Cell lysates were probed with an anti-PDE4A or an anti-p75NTR antibody (9651) to detect the expression levels of PDE4A and p75 receptors, respectively. Endogenous co-immunoprecipitation of p75NTR with PDE4A5 in primary CGNs was performed as in (A) (see e.g., FIG. 15B). Mapping of the p75NTR sites required for interaction with PDE4A5 (see e.g., FIG. 15C). Schematic diagram of HA-tagged p75NTR intracellular deletions. TM represents the transmembrane domain. DD represents the cytoplasmic death domain. PDE4A5 was co-transfected with HA-tagged p75NTR deletion constructs into 293 cells. Cell lysates were immunoprecipitated (IP) with an anti-HA antibody and probed with an anti-PDE4A antibody to detect co-precipitated PDE4A. Cell lysates were probed with an anti-PDE4A or an anti-p75NTR antibody (9651) to detect the expression levels of PDE4A and p75 receptors, respectively. IB, Immunoblot.

### Example 12

### Mapping the PDE4A4 Sequences that Interact with p75NTR

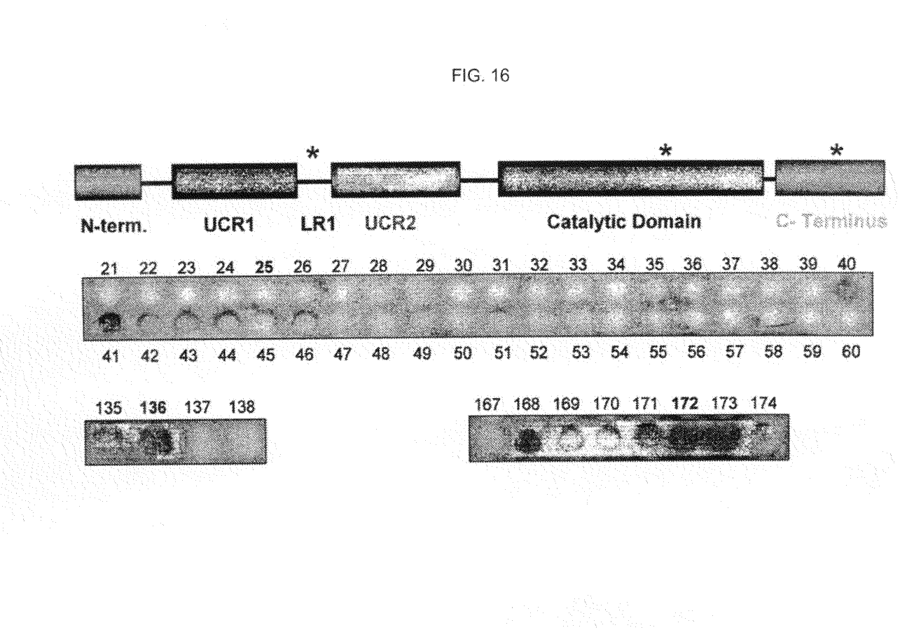

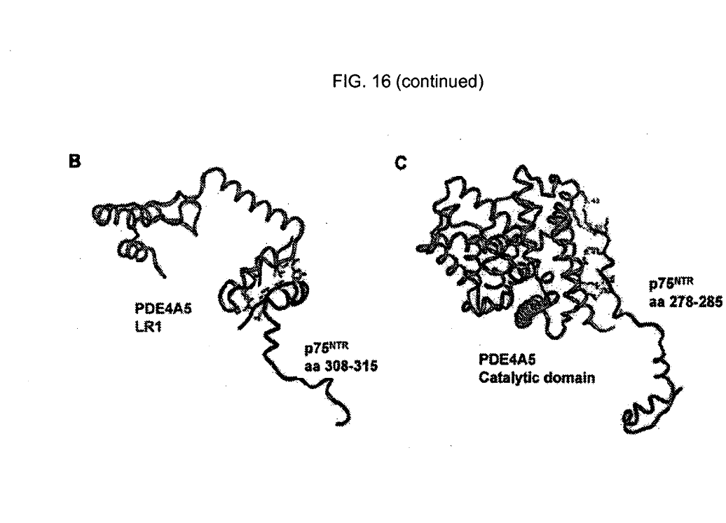

Peptide libraries were synthesized by automatic SPOT synthesis (see e.g., FIG. 16A). FIG. 16 depicts (A) Peptide libraries were synthesized by automatic SPOT synthesis. Synthetic overlapping peptides (twenty-five amino acids in length) were spotted on Whatman 50 membranes and overlaid with 10 μg/ml recombinant GST-p75NTR ICD. Bound recombinant GST-p75NTR ICD was detected using rabbit anti-GST followed by secondary anti-rabbit horseradish peroxidase antibody. This analysis revealed three distinct domains of PDE4A4 that interact with the intracellular domain of p75NTR: the LR1 domain (peptides 40 and 41, sequence SLLTNVPVPSNKRSPLGGPTPVCKATLSEE), the catalytic domain (peptides 135 and 136, sequence TLEDNRDWYYSAIRQSPSPPPEEESRGPGH), and the unique C-terminus (peptides 172 and 173, sequence KRACSACAGTFGEDTSALPAPGGGG SGGDP). (B&C) Models of the interacting sequences of PDE4A4 and p75NTR. (B) Computer simulated docking of the LR1 domain of PDE4A4 with the p75NTR ICD. (C) Computer simulated docking of the catalytic domain of PDE4A4 with the p75NTR ICD. In both the LR1 and the catalytic domain, the residues of PDE4A4 shown to interact with p75NTR ICD in silico are found to be within the same interacting sequences identified in vitro using peptide arrays and co-immunoprecipitation.

Synthetic overlapping peptides (twenty-five amino acids in length) were spotted on Whatman 50 membranes and overlaid with 10 μg/ml recombinant GST-p75NTR ICD. Bound recombinant GST-p75NTR ICD was detected using rabbit anti-GST followed by secondary anti-rabbit horseradish peroxidase antibody. This analysis revealed three distinct domains of PDE4A4 that interact with the intracellular domain of p75NTR: the LR1 domain (peptides 40 and 41, sequence SLLTNVPVPSNKRSPLGGPTPVCKATLSEE), the catalytic domain (peptides 135 and 136, sequence TLEDNRDWYYSAIRQSPSPPPEEESRGPGH), and the unique C-terminus (peptides 172 and 173, sequence KRACSACAGTFGEDTSALPAPGGGG SGGDP). Models of the interacting sequences of PDE4A4 and p75NTR (see e.g., FIGS. 16 B&C). Computer simulated docking of the LR1 domain of PDE4A4 with the p75NTR ICD (see e.g., FIG. 16B). Computer simulated docking of the catalytic domain of PDE4A4 with the p75NTR ICD (see e.g., FIG. 16C). In both the LR1 and the catalytic domain, the residues of PDE4A4 shown to interact with p75NTR ICD in silico are found to be within the same interacting sequences identified in vitro using peptide arrays and co-immunoprecipitation.

### Example 13

### Blocking the PDE4A-p75NTR Interaction Overcomes Myelin Inhibition of Neurite Outgrowth

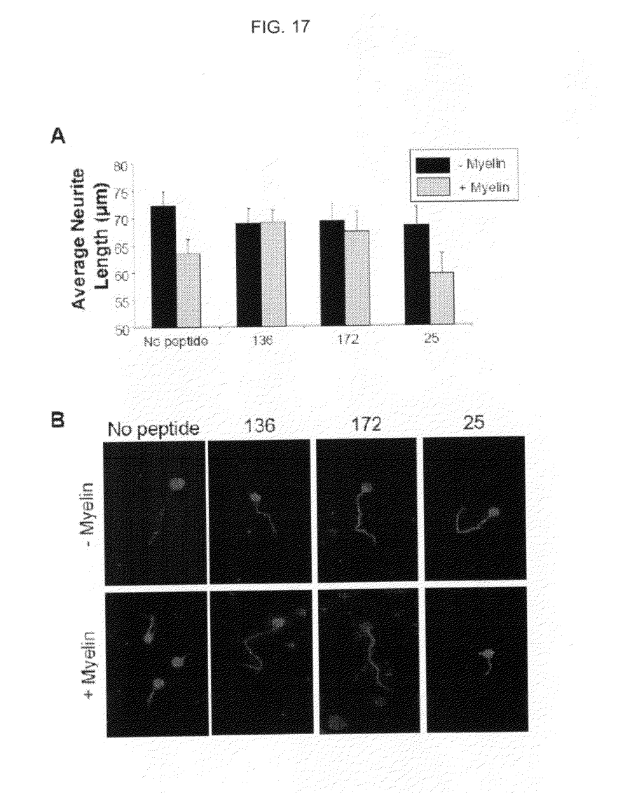

Two peptides designed to competitively inhibit the interaction between PDE4A4 and p75NTR were synthesized, as well as a negative control peptide (see e.g., FIG. 17). FIG. 17 depicts (A) We synthesized two peptides designed to competitively inhibit the interaction between PDE4A4 and p75NTR, as well as a negative control peptide. Each peptide was comprised of an 11 amino acid sequence taken from the HIV TAT protein (to confer cell permeability) fused to a PDE4A4 sequence. Peptide 136 (YGRKKRRQRRRRDWYYSAIRQSPSPPPEEESRGPGH; SEQ ID NO: 5) contained the catalytic domain interacting sequence, peptide 172 (YGRKKRRQRRRKRACSACAGTFGEDTSALPAPGGGG; SEQ ID NO: 6) was comprised of the unique C-terminal sequence, and the negative control peptide 25 (YGRKKRRQRRRSPLDSQASPGLVLHAGATTSQRRES) was derived from an N terminal sequence (partially contained within UCR1) that did not interact with p75NTR. We tested these peptides for their ability to overcome myelin inhibition of neurite outgrowth. Primary CGNs were plated on poly-D-lysine coated chamber slides and allowed to extend processes for 24 hrs in the presence or absence of myelin (1 μg/well). In addition to myelin treatment, cells were also treated with peptide 136, peptide 172, or negative control peptide 25. CGNs grown in the presence of myelin showed reduced neurite length compared to control cells (p<0.05). Treatment of CGNs with the 136 or 172 peptides prevented myelin inhibition of neurite outgrowth. The negative control peptide 25 did not overcome myelin inhibition of neurite outgrowth (p<0.05). (n=at least 100 neurites from each condition).

Each peptide was comprised of an 11 amino acid sequence taken from the HIV TAT protein (to confer cell permeability) fused to a PDE4A4 sequence. Peptide 136 (YGRKKRRQRRRRDWYYSAIRQSPSPPPEEESRGPGH; SEQ ID NO: 5) contained the catalytic domain interacting sequence, peptide 172 (YGRKKRRQRRRKRACSACAGTFGEDTSALPAPGGGG; SEQ ID NO: 6) was comprised of the unique C-terminal sequence, and the negative control peptide 25 (YGRKKRRQRRRSPLDSQASPGLVLHAGATTSQRRES; SEQ ID NO: 8) was derived from an N-terminal sequence (partially contained within UCR1) that did not interact with p75NTR. These peptides were tested for their ability to overcome myelin inhibition of neurite outgrowth. Primary CGNs were plated on poly-D-lysine coated chamber slides and allowed to extend processes for 24 hrs in the presence or absence of myelin (1 μg/well). In addition to myelin treatment, cells were also treated with peptide 136, peptide 172, or negative control peptide 25. CGNs grown in the presence of myelin showed reduced neurite length compared to control cells (p<0.05). Treatment of CGNs with the 136 or 172 peptides prevented myelin inhibition of neurite outgrowth. The negative control peptide 25 did not overcome myelin inhibition of neurite outgrowth (p<0.05). (n=at least 100 neurites from each condition)

### Example 14

### Interacting Sequences of PDE4A4

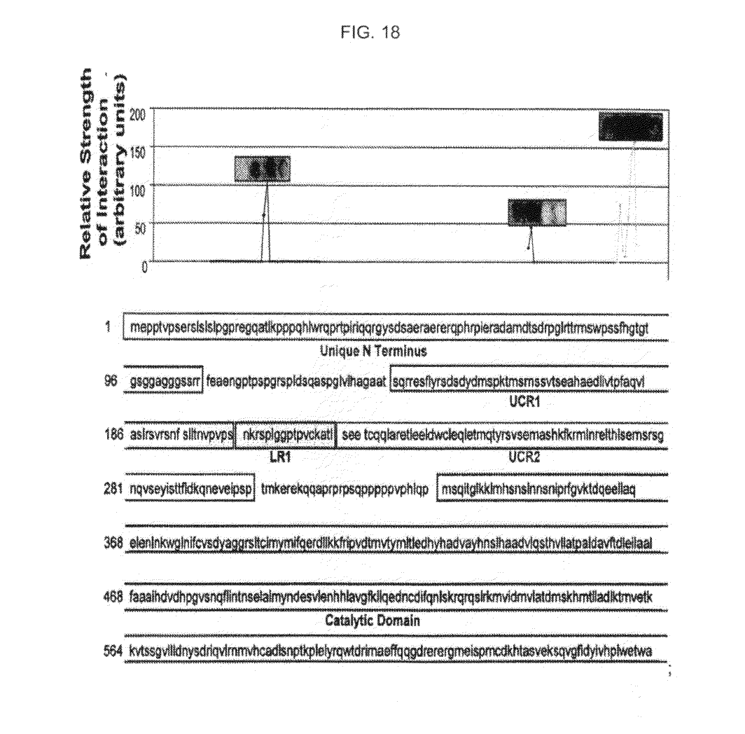

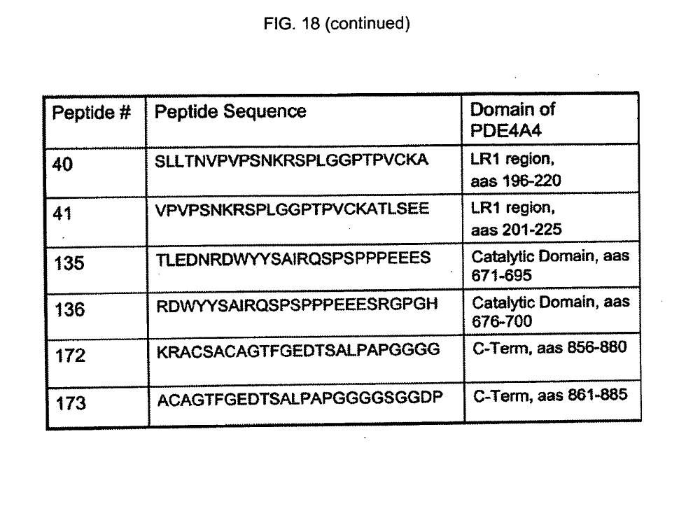

Quantitation of peptide array signal using densitomtry with NIH Scion Image software (see e.g., FIG. 18A). FIG. 18 depicts (A) Quantitation of peptide array signal using densitomtry with NIH Scion Image software. The C-terminal domain of PDE4A4 exhibited the strongest interaction with the p75NTR ICD, followed by the LR1 and catalytic domains. No significant interaction was observed in other domains of PDE4A4. (B) Sequence of PDE4A4 with domains delineated and interacting sequences highlighted in yellow. (C) Table of PDE4A4 sequences that were found to interact with p75NTR.

The C-terminal domain of PDE4A4 exhibited the strongest interaction with the p75NTR ICD, followed by the LR1 and catalytic domains. No significant interaction was observed in other domains of PDE4A4. Sequence of PDE4A4 with domains delineated and interacting sequences highlighted in yellow (see e.g., FIG. 18B). Table of PDE4A4 sequences that were found to interact with p75NTR (see e.g., FIG. 18C).

### Example 15

### Methodology

Animals, sciatic nerve crush, and induction of lung fibrosis p75NTR−/− mice (Lee et al., 1992) and tPA−/− mice (Carmeliet et al., 1994) were in C57B1/6 background and purchased from The Jackson Laboratory. Double p75NTR−/−tPA−/− mice were generated by crossing p75NTR−/− mice with tPA−/− mice. C57B1/6J mice were used as controls. Sciatic nerve crush was performed as described previously (Akassoglou et al., 2000). Lung fibrosis was induced as described previously (Chen et al., 2004). For the rolipram treatments, mice were administered 5 mg/kg rolipram (Calbiochem) before the LPS injection as described previously (Miotla et al., 1998). Mice were killed 4.5 h after LPS or saline administration. For rolipram treatment after sciatic nerve injury, mice were injected with rolipram (1 mg/kg) once daily for 8 d until tissue was harvested and processed for immunostaining.

Immunohistochemistry

Immunohistochemistry was performed as described in Akassoglou et al. (2002). Primary antibodies were sheep anti-human fibrin(ogen) (1:200; US Biologicals), rabbit anti-human tPA ( 1/300; Molecular Innovations), rabbit anti-p75NTR clone 9651, (1:1,000), goat anti-p75NTR ( 1/200; Santa Cruz Biotechnology, Inc.), rabbit anti-mouse PAI-1 (1:500; a gift from David Loskutoff, Scripps Research Institute, La Jolla, Calif.), and mouse anti-S100 (1:200; Neomarkers). For immunofluorescence, secondary antibodies were anti-rabbit FITC and anti-goat Cy3 (1:200; Jackson Immunochemicals). Images were acquired with an Axioplan II epifluorescence microscope (Carl Zeiss Microlmaging, Inc.) using dry Plan-Neofluar lenses using 10×0.3 NA, 20×0.5 NA, or 40×0.75 NA objectives equipped with Axiocam HRc digital camera and the Axiovision image analysis system.

Immunoblot

Immunoblot was performed as described previously (Akassoglou et al., 2002). Antibodies used were rabbit anti-p75NTR clones 9992 and 9651 (1:5,000), mouse anti-fibrin (1:500; Accurate Chemical & Scientific Corp.), rabbit anti-myosin (1:1,000; Sigma-Aldrich), rabbit anti-GAPDH (1:5,000; Abcam) and rabbit anti-PAI-1 (1:5,000; a gift of David Loskutoff). Quantification was performed on the Scion NIH Imaging Software. Fibrin precipitation and quantification from lung tissues was performed exactly as described previously (Ling et al., 2004).

Co-IP

Co-IP was performed as described previously (Khursigara et al., 1999). Cell lysates were prepared in 1% NP-40, 200 mM NaCl, 1 mM EDTA, and 20 mM Tris-HCl, pH 8.0. IP was performed with an anti-p75NTR antibody (9992) and immunoblot with anti-PDE4A, PDE4B, PDE4C, and PDE4D (Fabgennix). The co-IP buffer using NP-40 has been previously used to examine interactions of p75NTR with other intracellular proteins, such as TRAF-6 (Khursigara et al., 1999) and PKA (Higuchi et al., 2003). For mapping experiments, PDE4A5 cDNA was cotransfected with HA-tagged p75NTR deletion constructs into HEK293 cells. IP was performed with an anti-HA antibody (Cell Signaling). Cell lysates were probed with an anti-PDE4A or an anti-p75NTR antibody (9651). For co-IP experiments using recombinant proteins, equimolar amounts (2 μM) of purified recombinant MBP-PDE4A5 (O'Connell et al., 1996), MPB-PDE4A4 (McPhee et al., 1999), MBPPDE4D3 (Yarwood et al., 1999), and GST-p75NTR-ICD (Khursigara et al., 2001) were mixed in binding buffer (50 mM Tris-HCl, pH 7.5, 100 mM NaCl, 2 mM MgCl2, 1 mM DTT, 0.5% Triton X-100, and 0.5% BSA) and incubated for 1 h at 4° C. Washed glutathione-Sepharose beads were added according to the manufacturer's instructions for an additional hour. Beads were sedimented by centrifugation (10,000 g for 1 min) and washed three times. Proteins associated with the beads were eluted by boiling in loading buffer and separated by SDS-PAGE.

RT-PCR and Real-Time PCR

RT-PCR was performed as described previously (Akassoglou et al., 2002). Primers for tPA, uPA, and PAI-1 genes were used as described previously (Yamamoto and Loskutoff, 1996). Real-time PCR was performed using the Opticon DNA Engine 2 (MJ Research) and the Quantitect SYBR Green PCR kit (QIAGEN). Results were analyzed with Opticon 2 software using the comparative Ct method as described previously (Livak and Schmittgen, 2001). Data were expressed as ΔΔCt for the tPA gene normalized against GAPDH.

Quantification of tPA and uPA Activity

Quantification of tPA and uPA activity in SC and fibroblast in lysates and supernatants was performed according to the directions of the activity assay kits from American Diagnostica and Chemicon, respectively. To elevate cAMP cells were treated either with 2 mM db-cAMP (Sigma-Aldrich) or with 10 μM forskolin (Sigma-Aldrich) for 16 h. To block PKA activity, cells were treated with 200 nM KT5720 (Calbiochem). Induction with neurotrophins was performed using 100 ng/ml NGF and 50 ng/ml BDNF for 16 h before tPA assay.

Fibrin Degradation Assay

Coating with fibrin was prepared as described previously (Lansink et al., 1998). To quantitate fibrin degradation, the supernatant was aspirated and the remaining gel was weighed using an analytical balance. Decrease of gel weight corresponded to increased fibrin gel degradation.

Cell Culture and Transfections

Murine SCs were isolated as described previously (Syroid et al., 2000). NIH3T3 or HEK293 cells were cotransfected either with p75NTR FL, ICD or deletion constructs, and PDE4A5 cDNAs using Lipofectamine 2000 (Invitrogen) as described in the Results section. CGNs were isolated from P10 animals (Yamashita and Tohyama, 2003). CGNs were lysed immediately for co-IP, without plating. siRNA directed against p75NTR (Dharmacon; SMART Pool reagent, Cat. M-080041-00; Cat. M-009340) was transfected into SCs and NIH3T3p75NTR cells using Dharmafect (Dharmacon).

cAMP/PKA Assays

106 fibroblasts or 500,000 SCs were lysed in 0.1 N HCl solution and cAMP was measured using a competitive binding cAMP ELISA (Assay Designs). Cells were treated with 100 ng/ml PTX for 16 h. For inhibition of PDE activity, cells were treated for 16 h with 500 μM isobutyl methylxanthine (IBMX; Calbiochem), 18.7 μM 8-methoxymethyl-3-isobutyl-1-methylxanthine (PDE1 inhibitor; Calbiochem), 80 μM erythro-9-(2-Hydroxy-3-nonyl)adenine (PDE2 inhibitor; Calbiochem), 100 nM trequinsin (PDE3 inhibitor; Calbiochem), and 10 μM rolipram (PDE4 inhibitor; Calbiochem). Cells were treated with forskolin in the presence of the inhibitors for 1 h. Because these inhibitors specifically inhibit a PDE isoform and have no effect on the other PDE isoenzymes (Beavo and Reifsnyder, 1990), they are extensively used for the identification of the specific PDE isoform that is involved in different cellular functions. Induction with neurotrophins was performed using 100 ng/ml NGF or 50 ng/ml BDNF, 750 ng/ml of FcTrkB, or 1.35 ug/ml of Fcp75NTR for 1 h before cAMP assay. For the qualitative and quantitative PKA assay (Promega), cells were treated with 10 μM forskolin for 30 min, lysed in 1% NP-40 buffer with 150 mM NaCl, 50 mM Tris, and 1 mM EGTA, and protein concentration was determined using the Bradford Assay (Bio-Rad Laboratories). 1 μg was loaded into the PKA assay reaction mix according to the manufacturer's protocol (Promega).

In Situ Zymography

In situ zymographies were performed as described previously (Akassoglou et al., 2000). Quantification of in situ zymographies was performed by measuring the area of the lytic zone surrounding each nerve, and dividing that value by the area of the nerve. Images were collected after 8 h of incubation for the sciatic nerve and 4 h of incubation for the lung. For cell zymographies, cultures were washed four times with PBS/BSA and overlaid with 200 μl of Dulbecco's minimum essential medium containing 1% LMP agarose, 2.5% boiled nonfat milk, and 25 μg/ml human plasminogen. The overlay was allowed to harden, and plates were incubated in a cell culture incubator at 37° C. Pictures of lytic zones were taken using an inverted microscope under dark field (Carl Zeiss Microlmaging, Inc.).

Construction of pm-AKAR2.2 and PDE4A4ΔCT

For the construction of pm-AKAR2.2 the previously described cytoplasmic PKA sensor was used, AKAR2 (Zhang et al., 2005). pm-AKAR2.2 consists of a cDNA containing a FRET pair, monomeric enhanced cyan fluorescent protein (ECFP), and monomeric citrine (an optimized version of YFP), fused to forkhead associated domain 1 (FHA1) (Rad53p 22-162), and the PKA substrate sequence LRRATLVD via linkers. A206K mutations were incorporated to ECFP and Citrine by the QuikChange method (Stratagene). The C-terminal sequence from K-Ras K K K K K K S K T K C V I M was added to target the construct to the plasma membrane. For expression in mammalian cells, the chimaeric proteins were subcloned into a modified pcDNA3 vector (Invitrogen) behind a Kozak sequence as described previously (Zhang et al., 2005). For the generation of the PDE4A4δCT, PDE4A4 was subcloned into p3XFLAG-CMV-14 using plasmid pde46 (GenBank/EMBL/DDBJ accession no. L20965) as template from Met-1 to Iso-721 (McPhee et al., 1999). A forward (5′) primer containing a HindIII restriction site immediately 5′ to the initiating Met-1 (ATG) of PDE4A4 and a reverse primer designed to the DNA sequence ending at Iso-721 (ATA) with BamHI restriction site immediately 3′ to Iso-721 was used to amplify Met-1 to Iso-721. The C terminus was removed simply by amplifying from Iso-721 instead of the final codon at the end of the full-length PDE4A4B. The C-terminally truncated PDE4A4B was cloned in-frame with three FLAG (Asp-Tyr-Lys-Xaa-Xaa-Asp) epitopes (Asp-726, Asp-733 & Asp-740) after the BamHI restriction site, therefore at the C terminus of the now-truncated PDE4A4. The stop codon (TAG) after the FLAG epitopes is located immediately after Lys-747. This strategy generates a C-terminal truncate of PDE4A4 from 1-721.

FRET Imaging

NIH3T3 cells and NIH3T3p75NTR cells were transiently transfected with pm-AKAR2.2, AKAR3, or pm-AKAR3 (Allen and Zhang, 2006) and imaged within 24 h of transfection. Cells were rinsed once with HBSS (Cellgro) before imaging in HBSS in the dark at room temperature. An Axiovert microscope (Carl Zeiss Microimaging, Inc.) with a MicroMax digital camera (Roper-Princeton Instruments) and MetaFluor software (Universal Imaging Corp.) was used to acquire all images. Optical filters were obtained from Chroma Technologies. CFP and FRET images were taken at 15-s intervals. Dual emission ratio imaging used a 420/20-nm excitation filter, a 450-nm dichroic mirror and a 475/40-nm or 535/25-nm emission filter for CFP and FRET, respectively. Excitation and emission filters were switched in filter wheels (Lambda 10-2; Sutter Instrument Co.).

Peptide Array Mapping

Peptide libraries were synthesized by automatic SPOT synthesis (Frank, 2002). Synthetic overlapping peptides (25 amino acids in length) were spotted on Whatman 50 cellulose membranes according to standard protocols by using Fmoc-chemistry with the AutoSpot Robot ASS 222 (Intavis Bioanalytical Instruments AG). Membranes were overlaid with 10 μg/ml recombinant GST-p75NTR ICD. Bound recombinant GST-p75NTR ICD (Khursigara et al., 2001) was detected using rabbit anti-GST (1:2,000; GE Healthcare) followed by secondary anti-rabbit horseradish peroxidase antibody (1:2,500; Dianova). Alanine scanning was performed as described previously (Bolger et al., 2006).

Statistics

Statistical significance was calculated using JMP2 Software by unpaired t test for isolated pairs or by analysis of variance (one-way ANOVA) for multiple comparisons. Data are shown as the mean±SEM.

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

Citation of a reference herein shall not be construed as an admission that such is prior art to the present invention. Specifically intended to be within the scope of the present invention, and incorporated herein by reference in its entirety, is the following publication: B. D. Sachs, et al., p75 neurotrophin receptor regulates tissue fibrosis through inhibition of plasminogen activation via a PDE4/cAMP/PKA pathway, The Journal of Cell Biology, Vol. 177, No. 6, 1119-1132 (2007).

Other references referred to herein include:
Adams, R. A., M. Passino, B. D. Sachs, T. Nuriel, and K. Akassoglou. 2004. Fibrin mechanisms and functions in nervous system pathology. Mol. Interv. 4:163-176.
Akassoglou, K., K. W. Kombrinck, J. L. Degen, and S. Strickland. 2000. Tissue plasminogen activator-mediated fi brinolysis protects against axonal degeneration and demyelination after sciatic nerve injury. J. Cell Biol. 149:1157-1166.
Akassoglou, K., W.-M. Yu, P. Akpinar, and S. Strickland. 2002. Fibrin inhibits peripheral nerve regeneration by arresting Schwann cell differentiation. Neuron. 33:861-875.
Allen, M. D., and J. Zhang. 2006. Subcellular dynamics of protein kinase A activity visualized by FRET-based reporters. Biochem. Biophys. Res. Commun. 348:716-721.
Barber, R., G. S. Baillie, R. Bergmann, M. C. Shepherd, R. Sepper, M. D. Houslay, and G. V. Heeke. 2004. Differential expression of PDE4 cAMP phosphodiesterase isoforms in inflammatory cells of smokers with COPD, smokers without COPD, and nonsmokers. Am. J. Physiol. Lung Cell. Mol. Physiol. 287:L332-L343.
Baron, P., E. Scarpini, S. Pizzul, F. Zotti, G. Conti, D. Pleasure, and G. Scarlato. 1997. Immunocytochemical expression of human muscle cell p75 neurotrophin receptor is down-regulated by cyclic adenosine 3′,5′-monophosphate. Neurosci. Lett. 234:79-82.
Beattie, M. S., A. W. Harrington, R. Lee, J. Y. Kim, S. L. Boyce, F. M. Longo, J. C. Bresnahan, B. L. Hempstead, and S. O. Yoon. 2002. ProNGF induces p75-mediated death of oligodendrocytes following spinal cord injury. Neuron. 36:375-386.
Beavo, J. A., and D. H. Reifsnyder. 1990. Primary sequence of cyclic nucleotide phosphodiesterase isozymes and the design of selective inhibitors. Trends Pharmacol. Sci. 11:150-155.
Bolger, G. B., G. S. Baillie, X. Li, M. J. Lynch, P. Herzyk, A. Mohamed, L. H. Mitchell, A. McCahill, C. Hundsrucker, E. Klussmann, et al. 2006. Scanning peptide array analyses identify overlapping binding sites for the signalling scaffold proteins, beta-arrestin and RACK1, in cAMP-specific phosphodiesterase PDE4D5. Biochem. J. 398:23-36.
Brunton, L. L. 2003. PDE4: arrested at the border. Sci. STKE. 2003:PE44. Bugge, T. H., K. W. Kombrinck, M. J. Flick, C. C. Daugherty, M. J. Danton, and J. L. Degen. 1996. Loss of fibrinogen rescues mice from the pleiotropic effects of plasminogen deficiency. Cell. 87:709-719.
Carmeliet, P., L. Schoonjans, L. Kieckens, B. Ream, J. Degen, R. Bronson, R. De Vos, J. J. van den Oord, D. Collen, and R. C. Mulligan. 1994. Physiological consequences of loss of plasminogen activator gene function in mice. Nature. 368:419-424.
Chao, M. V. 2003. Neurotrophins and their receptors: a convergence point for many signalling pathways. Nat. Rev. Neurosci. 4:299-309.
Chen, D., K. Giannopoulos, P. G. Shiels, Z. Webster, J. H. McVey, G. Kemball-Cook, E. Tuddenham, M. Moore, R. Lechler, and A. Dorling. 2004. Inhibition of intravascular thrombosis in murine endotoxemia by targeted expression of hirudin and tissue factor pathway inhibitor analogs to activated endothelium. Blood. 104:1344-1349.
Chemy, J. A., and R. L. Davis. 1999. Cyclic AMP phosphodiesterases are localized in regions of the mouse brain associated with reinforcement, movement, and affect. J. Comp. Neurol. 407:287-301.
Cosgaya, J. M., J. R. Chan, and E. M. Shooter. 2002. The neurotrophin receptor p75NTR as a positive modulator of myelination. Science. 298:1245-1248.
Degen, J. L., A. F. Drew, J. S. Palumbo, K. W. Kombrinck, J. A. Bezerra, M. J. Danton, K. Holmback, and T. T. Suh. 2001. Genetic manipulation of fi brinogen and fi brinolysis in mice. Ann. N.Y. Acad. Sci. 936:276-290.
Dowling, P., X. Ming, S. Raval, W. Husar, P. Casaccia-Bonnefi I, M. Chao, S. Cook, and B. Blumberg. 1999. Up-regulated p75NTR neurotrophin receptor on glial cells in MS plaques. Neurology. 53:1676-1682.
Filbin, M. T. 2003. Myelin-associated inhibitors of axonal regeneration in the adult mammalian CNS. Nat. Rev. Neurosci. 4:703-713.
Fiumelli, H., D. Jabaudon, P. J. Magistretti, and J.-L. Martin. 1999. BDNF stimulates expression, activity and release of tissue-type plasminogen activator in mouse cortical neurons. Eur. J. Neurosci. 11:1639-1646.
Frank, R. 2002. The SPOT-synthesis technique. Synthetic peptide arrays on membrane supports—principles and applications. J. Immunol. Methods. 267:13-26.
Gao, Y., E. Nikulina, W. Mellado, and M. T. Filbin. 2003. Neurotrophins elevate cAMP to reach a threshold required to overcome inhibition by MAG through extracellular signal-regulated kinase-dependent inhibition of phosphodiesterase. J. Neurosci. 23:11770-11777.
Gretarsdottir, S., G. Thorleifsson, S. T. Reynisdottir, A. Manolescu, S. Jonsdottir, T. Jonsdottir, T. Gudmundsdottir, S. M. Bjarnadottir, O. B. Einarsson, H. M. Gudjonsdottir, et al. 2003. The gene encoding phosphodiesterase 4D confers risk of ischemic stroke. Nat. Genet. 35:131-138.
Herrmann, J. L., D. G. Menter, J. Hamada, D. Marchetti, M. Nakajima, and G. L. Nicolson. 1993. Mediation of NGF-stimulated extracellular matrix invasion by the human melanoma low-affinity p75 neurotrophin receptor: melanoma p75 functions independently of trkA. Mol. Biol. Cell. 4:1205-1216.
Higuchi, H., T. Yamashita, H. Yoshikawa, and M. Tohyama. 2003. PKA phosphorylates the p75 receptor and regulates its localization to lipid rafts. EMBO J. 22:1790-1800.
Houslay, M. D., and D. R. Adams. 2003. PDE4 cAMP phosphodiesterases: modular enzymes that orchestrate signalling cross-talk, desensitization and compartmentalization. Biochem. J. 370:1-18.
Houslay, M. D., P. Schafer, and K. Y. Zhang. 2005. Keynote review: phosphodiesterase-4 as a therapeutic target. Drug Discov. Today. 10:1503-1519.
Hsu, K. C., and M. V. Chao. 1993. Differential expression and ligand binding properties of tumor necrosis factor receptor chimeric mutants. J. Biol. Chem. 268:16430-16436.
Idell, S. 2003. Coagulation, fi brinolysis, and fi brin deposition in acute lung injury. Crit. Care Med. 31:S213-S220.
Kawasaki, T., M. Dewerchin, H. R. Lijnen, I. Vreys, J. Vermylen, and M. F. Hoylaerts. 2001. Mouse carotid artery ligation induces platelet-leukocytedependent luminal fi brin, required for neointima development. Circ. Res. 88:159-166.
Khursigara, G., J. R. Orlinick, and M. V. Chao. 1999. Association of the p75 neurotrophin receptor with TRAF6. J. Biol. Chem. 274:2597-2600.
Khursigara, G., J. Bertin, H. Yano, H. Moffett, P. S. DiStefano, and M. V. Chao. 2001. A prosurvival function for the p75 receptor death domain mediated via the caspase recruitment domain receptor-interacting protein 2. J. Neurosci. 21:5854-5863.
Kraemer, R. 2002. Reduced apoptosis and increased lesion development in the flow-restricted carotid artery of p75(NTR)-null mutant mice. Circ. Res. 91:494-500.
Lansink, M., P. Koolwijk, V. van Hinsbergh, and T. Kooistra. 1998. Effect of steroid hormones and retinoids on the formation of capillary-like tubular structures of human microvascular endothelial cells in fi brin matrices is related to urokinase expression. Blood. 92:927-938.
Lee, K. F., E. Li, L. J. Huber, S. C. Landis, A. H. Sharpe, M. V. Chao, and R. Jaenisch. 1992. Targeted mutation of the gene encoding the low affinity NGF receptor p75 leads to deficits in the peripheral sensory nervous system. Cell. 69:737-749.
Lee, R., P. Kermani, K. K. Teng, and B. L. Hempstead. 2001. Regulation of cell survival by secreted proneurotrophins. Science. 294:1945-1948.
Lehnart, S. E., X. H. Wehrens, S. Reiken, S. Warder, A. E. Belevych, R. D. Harvey, W. Richter, S. L. Jin, M. Conti, and A. R. Marks. 2005. Phosphodiesterase 4D deficiency in the ryanodine-receptor complex promotes heart failure and arrhythmias. Cell. 123:25-35.
Lemke, G., and M. Chao. 1988. Axons regulate Schwann cell expression of the major myelin and NGF receptor genes. Development. 102:499-504. Lijnen, H. R. 2001. Elements of the fibrinolytic system. Ann. N.Y. Acad. Sci. 936:226-236.
Ling, Q., A. T. Jacovina, A. Deora, M. Febbraio, R. Simantov, R. L. Silverstein, B. Hempstead, W. H. Mark, and K. A. Hajjar. 2004. Annexin II regulates fi brin homeostasis and neoangiogenesis in vivo. J. Clin. Invest. 113:38-48.
Livak, K. J., and T. D. Schmittgen. 2001. Analysis of relative gene expression data using real-time quantitative PCR and the 2(-Delta Delta C(T)) method. Methods. 25:402-408.
Lomen-Hoerth, C., and E. M. Shooter. 1995. Widespread neurotrophin receptor expression in the immune system and other normeuronal rat tissues. J. Neurochem. 64:1780-1789.
McPhee, I., S. J. Yarwood, G. Scotland, E. Huston, M. B. Beard, A. H. Ross, E. S. Houslay, and M. D. Houslay. 1999. Association with the SRC family tyrosyl kinase LYN triggers a conformational change in the catalytic region of human cAMP-specific phosphodiesterase HSPDE4A4B. Consequences for rolipram inhibition. J. Biol. Chem. 274:11796-11810.
Medcalf, R. L., M. Ruegg, and W. D. Schleuning. 1990. A DNA motif related to the cAMP-responsive element and an exon-located activator protein-2 binding site in the human tissue-type plasminogen activator gene promoter cooperate in basal expression and convey activation by phorbol ester and cAMP. J. Biol. Chem. 265:14618-14626.
Millar, J. K., B. S. Pickard, S. Mackie, R. James, S. Christie, S. R. Buchanan, M. P. Malloy, J. E. Chubb, E. Huston, G. S. Baillie, et al. 2005. DISC1 and PDE4B are interacting genetic factors in schizophrenia that regulate cAMP signaling. Science. 310:1187-1191.
Miotla, J. M., M. M. Teixeira, and P. G. Hellewell. 1998. Suppression of acute lung injury in mice by an inhibitor of phosphodiesterase type 4. Am. J. Respir. Cell Mol. Biol. 18:411-420.
Nikulina, E., J. L. Tidwell, H. N. Dai, B. S. Bregman, and M. T. Filbin. 2004. The phosphodiesterase inhibitor rolipram delivered after a spinal cord lesion promotes axonal regeneration and functional recovery. Proc. Natl. Acad. Sci. USA. 101:8786-8790.
O'Connell, J. C., J. F. McCallum, I. McPhee, J. Wakefield, E. S. Houslay, W. Wishart, G. Bolger, M. Frame, and M. D. Houslay. 1996. The SH3 domain of Src tyrosyl protein kinase interacts with the N-terminal splice region of the PDE4A cAMP-specific phosphodiesterase RPDE-6 (RNPDE4A5). Biochem. J. 318(Pt 1):255-261.
Park, J. A., J. Y. Lee, T. A. Sato, and J. Y. Koh. 2000. Co-induction of p75NTR and p75NTR-associated death executor in neurons after zinc exposure in cortical culture or transient ischemia in the rat. J. Neurosci. 20:9096-9103.
Passino, M. A., R. A. Adams, S. L. Sikorski, and K. Akassoglou. 2007. Regulation of hepatic stellate cell differentiation by the neurotrophin receptor p75NTR. Science. 315:1853-1856.
Perry, S. J., G. S. Baillie, T. A. Kohout, I. McPhee, M. M. Magiera, K. L. Ang, W. E. Miller, A. J. McLean, M. Conti, M. D. Houslay, and R. J. Lefkowitz. 2002. Targeting of cyclic AMP degradation to beta 2-adrenergic receptors by beta-arrestins. Science. 298:834-836.
Qian, Z., M. E. Gilbert, M. A. Colicos, E. R. Kandel, and D. Kuhl. 1993. Tissueplasminogen activator is induced as an immediate-early gene during seizure, kindling and long-term potentiation. Nature. 361:453-457.
Rabizadeh, S., J. Oh, L. T. Zhong, J. Yang, C. M. Bitler, L. L. Butcher, and D. E. Bredesen. 1993. Induction of apoptosis by the low-affinity NGF receptor. Science. 261:345-348.
Reichardt, L. F. 2006. Neurotrophin-regulated signalling pathways. Philos. Trans. R. Soc. Lond. B Biol. Sci. 361:1545-1564.
Renz, H., S. Kerzel, and W. A. Nockher. 2004. The role of neurotrophins in bronchial asthma: contribution of the pan-neurotrophin receptor p75. Prog. Brain Res. 146:325-333.
Ricci, A., L. Felici, S. Mariotta, F. Mannino, G. Schmid, C. Terzano, G. Cardillo, F. Amenta, and E. Bronzetti. 2004. Neurotrophin and neurotrophin receptor protein expression in the human lung. Am. J. Respir. Cell Mol. Biol. 30:12-19.
Roux, P. P., A. L. Bhakar, T. E. Kennedy, and P. A. Barker. 2001. The p75 neurotrophin receptor activates Akt (protein kinase B) through a phosphatidylinositol 3-kinase-dependent pathway. J. Biol. Chem. 276:23097-23104.
Samson, A. L., and R. L. Medcalf. 2006. Tissue-type plasminogen activator: a multifaceted modulator of neurotransmission and synaptic plasticity. Neuron. 50:673-678.
Santell, L., and E. Levin. 1988. Cyclic AMP potentiates phorbol ester stimulation of tissue plasminogen activator release and inhibits secretion of plasminogen activator inhibitor-1 from human endothelial cells. J. Biol. Chem. 263:16802-16808.
Savoy, J. D., D. M. Brass, K. G. Berman, E. McElvania, and D. A. Schwartz. 2003. Fibrinolysis in LPS-induced chronic airway disease. Am. J. Physiol. Lung Cell. Mol. Physiol. 285:L940-L948.
Siconolfi, L. B., and N. W. Seeds. 2001. Mice lacking tPA, uPA, or plasminogen genes showed delayed functional recovery after sciatic nerve crush. J. Neurosci. 21:4348-4355.
Song, X. Y., F. H. Zhou, J. H. Zhong, L. L. Wu, and X. F. Zhou. 2006. Knockout of p75(NTR) impairs re-myelination of injured sciatic nerve in mice. J. Neurochem. 96:833-842.
Syroid, D. E., P. J. Maycox, M. Soilu-Hanninen, S. Petratos, T. Bucci, P. Burrola, S. Murray, S. Cheema, K. F. Lee, G. Lemke, and T. J. Kilpatrick. 2000. Induction of postnatal Schwann cell death by the low-affinity neurotrophin receptor in vitro and after axotomy. J. Neurosci. 20:5741-5747.
Taniuchi, M., H. B. Clark, and E. M. Johnson Jr. 1986. Induction of nerve growth factor receptor in Schwann cells after axotomy. Proc. Natl. Acad. Sci. USA. 83:4094-4098.
Teng, K. K., and B. L. Hempstead. 2004. Neurotrophins and their receptors: signaling trios in complex biological systems. Cell. Mol. Life Sci. 61:35-48. Walikonis, R. S., and J. F. Poduslo. 1998. Activity of cyclic AMP phosphodiesterases and adenylyl cyclase in peripheral nerve after crush and permanent transection injuries. J. Biol. Chem. 273:9070-9077.
Wang, S., P. Bray, T. McCaffrey, K. March, B. L. Hempstead, and R. Kraemer. 2000. p75(NTR) mediates neurotrophin-induced apoptosis of vascular smooth muscle cells. Am. J. Pathol. 157:1247-1258.
Yamamoto, K., and D. J. Loskutoff. 1996. Fibrin deposition in tissues from endotoxin-treated mice correlates with decreases in the expression of urokinase-type but not tissue-type plasminogen activator. J. Clin. Invest. 97:2440-2451.
Yamashita, T., and M. Tohyama. 2003. The p75 receptor acts as a displacement factor that releases Rho from Rho-GDI. Nat. Neurosci. 6:461-467. Yamashita, T., K. L. Tucker, and Y. A. Barde. 1999. Neurotrophin binding to the p75 receptor modulates Rho activity and axonal outgrowth. Neuron. 24:585-593.
Yarwood, S. J., M. R. Steele, G. Scotland, M. D. Houslay, and G. B. Bolger. 1999. The RACK1 signaling scaffold protein selectively interacts with the cAMP-specific phosphodiesterase PDE4D5 isoform. J. Biol. Chem. 274:14909-14917.
Zhang, J., C. J. Hupfeld, S. S. Taylor, J. M. Olefsky, and R. Y. Tsien. 2005. Insulin disrupts beta-adrenergic signalling to protein kinase A in adipocytes. Nature. 437:569-573.
Zorick, T. S., and G. Lemke. 1996. Schwann cell differentiation. Curr. Opin. Cell Biol. 8:870-876.

What is claimed is:

1. A method of treating a condition resulting from PDE4A4/5-mediated cAMP degradation, the method comprising administering to a subject in need thereof a therapeutically effective amount of an agent that disrupts the interaction between PDE4A4/5 and p75 neurotropin receptor (p75NTR).

2. A method according to claim 1, wherein the condition is a pulmonary disease or nerve injury.

3. A method according to claim 2, wherein the condition is COPD or spinal cord injury.

4. A method according to claim 1, wherein the agent comprises an isolated polypeptide comprising a sequence at least 80% identical to a LR1, catalytic, or C-terminus subunit of PDE4A4 and having an ability to specifically block the molecular interaction between p75NTR and PDE4A4/5.

5. A method according to claim 1, wherein the agent comprises an isolated polypeptide comprising SEQ ID NO: 2, SEQ ID NO: 3, SEQ ID NO: 4, SEQ ID NO: 5, SEQ ID NO: 6, or SEQ ID NO: 7, or a variant at least 80% identical thereof, and having an ability to specifically block the molecular interaction between p75NTR and PDE4A4/5.

6. An isolated polypeptide comprising a sequence at least 80% identical to a LR1, catalytic, or C-terminus subunit of PDE4A4 and having an ability to specifically block the molecular interaction between p75NTR and PDE4A4/5.

7. An isolated polypeptide according to claim 6, wherein the polypeptide specifically binds amino acid C862.

8. An isolated polypeptide comprising SEQ ID NO: 2, SEQ ID NO: 3, SEQ ID NO: 4, SEQ ID NO: 5, SEQ ID NO: 6, or SEQ ID NO: 7, or a variant at least 80% identical thereof, and having an ability to specifically block the molecular interaction between p75NTR and PDE4A4/5.

9. A method of screening an agent for treating a disease resulting from PDE4A4/5-mediated cAMP degradation, the method comprising:
providing a cell that stably expresses PDE4A4/5 and p75NTR;
administering a candidate agent to the cell;
measuring a level of PDE4A4/5-p75NTR complex in the cell; and
determining whether the candidate agent decreases the level of PDE4A4/5-p75NTR complex in the cell.

10. A method of screening an agent for treating a disease resulting from PDE4A4/5-mediated cAMP degradation, the method comprising:
providing PDE4A4/5 and p75NTR;
contacting a candidate agent, PDE4A4/5, and p75NTR;
measuring a level of PDE4A4/5-p75NTR complex; and
determining whether the candidate agent decreases the level of PDE4A4/5-p75NTR complex.
